(19) United States
(12) Patent Application Publication
(10) Pub. No.: US 2009/0041840 A1
(43) Pub. Date: Feb. 12, 2009

(54) Positive wakeup pharmaceutical sleep system with compatible pre-bedtime administration

(76) Inventor: William J. Ayala, Tampa, FL (US)

Correspondence Address: Robert F. Frijouf;FRIJOUF, RUST & PYLE, P.A., 201 East Davis Boulevard, Tampa, FL, 33606, US

(21) Appl. No.: 12/157,900

(22) Filed: Jun. 13, 2008

Related U.S. Application Data
(63) Continuation of application No. 10/633,460, filed on Aug. 4, 2003, now abandoned.

(51) Int. Cl. A61K 9/52; A61K 9/24; A61K 47/38; A61K 47/32; A61K 47/34; A61K 31/437; A61K 31/4045; A61K 31/137; A61K 31/519; A61K 31/445

(52) U.S. Cl. 424/457; 424/471; 514/781; 514/772.4; 514/772.3; 514/299; 514/415; 514/654; 514/259.3; 514/317

(57) ABSTRACT

A novel sleep regulating pharmaceutical formulation is introduced, typically implementing two principal drugs having actions which are reversive to one another, yet incorporated into a unitary solid dosage, and prepared for oral administration before bedtime. Usually, structure is configured to initially release a calmative or other sleep-compatible substance by prompt dissolution. The initial release is followed by a specific period of delay, which in basic formulations entails no release of any drug, and which allows a nominal interval of sleep. At the terminus of the delay, a final agent is released to induce wakeup. Incorporation of agents of opposite action within a unitary dosage form renders utility which is uniquely appropriate to the invention. In a preferred embodiment, delay of release and final delivery of wakeup agent are arranged by a dialysis membrane which eventually bursts as a result of osmotic pressure generated by a hydrophilic core.

### BACKGROUND OF THE INVENTION

This invention pertains to regulation of sleep chronology by pharmaceutical formulation. More particularly, the invention relates to a delayed-release combination, for administration by mouth before bedtime, which helps the patient both in falling asleep and in subsequently awakening. Arrangement within a single dosage form of reversive agents substantiates utility which is uniquely congruous with the purpose and objects of the concept. Additional benefits of the medication include improved alertness and vigor in the early hours following wakeup.

In the science of optimizing sleep, one aspect which has been virtually ignored is assistance in timely awakening by means other than external sensory stimulation from mechanical devices. It is well known that a significant segment of the population suffers from various sleep disorders, the suffering being consequently compounded by difficulty in waking up. More often than not, the sequence is followed by poor efficiency and productivity during the workday. Health science and medicine have long been concerned with these problems. Additional interest has recently been generated by the personal effectiveness and executive development fields. Regardless of field, the representative parties unanimously agree that, in any arena of human endeavor, even the simplest of daily tasks are impossible until a person wakes up and gets out of bed. Hence, punctual arousal could be deemed the single most important event of the day. The gravity of this crucial factor of health and time management can be elucidated by considering the circumstances of persons being treated for health conditions such as depression. It is not unusual for these patients to find that their difficulties are tragically snowballing if, due to a feeble start, they are late to arrive at work and subsequently lose their jobs. Accordingly, for individuals needing to maximize their punctuality, no superlative could overstate the advantage of awakening with efficiency. Furthermore, control of this fundamental aspect of the sleep cycle may lead to greater productivity sooner in the workday, thereby allowing persons having sleep dysfunction to relax earlier in the evening, and thus reducing incidence of insomnia. Moreover, certain indispensable activities of early waking hours require optimal alertness for a person to conduct them with a margin of safety. Motor vehicle operators, for instance, can seriously endanger themselves and others if, shortly after getting out of bed, they begin driving in a soporose mode.

The other important aspect of sleep optimization, that of transition of state of consciousness from waking to sleep, has been addressed extensively, with the most realistic success being accomplished by pharmaceutical tranquilizers. Of course, these medications are known to most everyone. It should be recognized, however, that such calmative drugs have never previously been integrated into one single device together with agents for the aforementioned major aspect, i.e.—impulsion of awakening.

### BACKGROUND

### Dysfunction of the Sleep Cycle, and Associated Socio-Economic Problems

### Sleep Disorders

The International Classification of Sleep Disorders (ICSD), published by the American Sleep Disorders Association (ASDA), lists as many as 70 sleep disorders (SDs). Those bearing strongly upon discussion here include the insomnias, hypersomnias, and certain neurological conditions and syndromes.

The original insomnia classification system had three general types: initial insomnia (difficulty in falling asleep), middle insomnia (difficulty in remaining asleep), and terminal insomnia (waking too early). Recent classifications identify at least a dozen specific insomnias.

Those which are germane include:

Idiopathic Insomnia (Chronic Insomnia, Primary Insomnia)—A lifelong inability to obtain adequate sleep that is presumably brought about by an abnormality of the neurological control of the sleep-wake system. The sleeping difficulty may be manifest as initial insomnia, middle insomnia, or both.

Adjustment Sleep Disorder (a/k/a Transient Insomnia)—Represents sleep disturbance temporally related to stress, conflict, or environmental change causing emotional agitation. Very common; all people are subjected to situational occurrences of insomnia. A particular form of Adjustment Sleep Disorder is:

Time Zone Change Syndrome (Jet Lag)—Consists of varying degrees of initial insomnia and/or middle insomnia, generally with consequent difficulty in waking up, and succeeded by excessive sleepiness with decrements in subjective daytime alertness and performance, following rapid travel across multiple time zones.

### Hypersomnias Most Relevant Include:

Idiopathic Hypersomnia (Primary Hypersomnia, Chronic Hypersomnia)—is characterized by excessive sleepiness of at least one month's duration, evidenced by near-daily diurnal sleep episodes, excessive naps, or abnormally prolonged sleep intervals. Sufferers do not wake refreshed, and may display signs of “sleep drunkenness,” or great difficulty making the transition from sleep to wakefulness.

Other cardinal sleep disorders include:

Delayed Sleep Phase Syndrome (DSPS)—A disorder in which the major sleep period is delayed in relation to the desired clock time resulting in symptoms of sleep onset insomnia (initial insomnia), or impedance to awakening at the desired time. Commonly, both problems are exhibited, sequence of cause and effect being uncertain.

Sleep Paralysis (Postdormital Paralysis)—consists of a period of inability to perform voluntary movements, especially upon awakening.

### Sleep Statistics

The marvelous “Sleep In America” polls, conducted by the National Sleep Foundation (NSF) in collaboration with The Gallup Organization, compiled statistics from 1991 through the present. The collected data indicate that the number of people who admit to some form of insomnia increased from 60 million to over 110 million. An average of 23% of adults had difficulty falling asleep at least a few nights a week, with an increase to 25% in 2002, indicating the problem may occur persistently in 32 million of the adult population. The prevalence has been even more common in younger adults 18-29 years old.

Consequences of insomnia are manifest. The NSF year 2000 poll discovered that for a variety of reasons including insomnia, nearly one out of four adults (24%) had trouble getting up for work two or more workdays per week. Conspicuously, more than one-third (36%) of younger adults 18-29 years old reported this difficulty.

One-half of U.S. adults (46%, 52%, 52%), in poll years 2000, 2001 and 2002, responded that they need an alarm clock to wake up four or more mornings a week. And, despite alarm clock usage, practically one out of seven adults (14%) in 2000 said that they were occasionally or frequently late to work due to sleepiness. This result of sleep deficiency is most often experienced by younger adults (22% of 18-29 year-olds).

### Traffic Accidents

According to the NHTSA, of 6,394,000 motor vehicle accidents reported by police in the U.S.A. during the year 2000, 37,409 crashes resulted in fatalities, 2,070,000 crashes caused injuries, and 4,286,000 caused property damage only. Of the total number of crashes 25,492 drivers were killed, accounting for 61 percent of the fatalities reported for the year (DOT HS 809 329).

Economic Detriment of MV Crashes—The NHTSA report, “Economic Impact of MV Crashes,” (DOT HS 809 446), released May 2002, indicates a total cost from traffic crashes in 2000 of $230.6 billion. Surprisingly, lost productivity accounted for the broadest section of macroeconomic effect, totaling $81.2 billion (35.2% of overall negative economics). Medical costs were responsible for $32.6 billion (14.1%), while property damage losses were $59.0 billion (25.6%). Public revenues paid for roughly 9 percent of all motor vehicle crash costs, gouging tax payers $21 billion in 2000, the equivalent of over $200 in added taxes for every U.S. household.

As stated by the FHWA's Table DL-20, “Distribution Of Licensed Drivers 2000,” the total of licensed U.S. drivers, all ages, was 190,625,023. There were 29,230,000 licensed drivers aged 25 years old or younger (15.3%). For this age group, 3,364,000 drivers (29.7%) were involved in some type of MV accident, double in ratio to licensed drivers. Of those in accidents, 15,400 were involved in mortalities (27.0%). Among the fatal wrecks, 6,770 drivers 25 or under were killed in 2000 (26.6%). The estimated economic cost of police-reported crashes, any degree of severity, involving drivers of this age bracket, was $68.5 billion.

From interpolation of raw data available from the FHWA, we have created a separate age grouping, i.e.—18-29 year old drivers, so as to enable a comparison to NSF poll statistics. Per Table DL-20, there were 38,658,000 licensed drivers aged 18 through 29, amounting to 20.3% of all licensed drivers in the USA.

As publicized in Table 63 of DOT HS 809 337, drivers 18-29 involved in all crashes were approximately 3,592,400 or 32%. Also, 657,300 (32%) of all drivers injured fell into this age group. The total for drivers 18-29 years old involved in fatalities was 16,500, or 29% of all drivers in fatal crashes, almost 1.5:1 in ratio to licensed drivers. Drivers killed in 2000, 18-29 years old, totaled 7,069, or 28% of all driver deaths. The estimated economic cost of police-reported crashes, any degree of severity, involving drivers of this age bracket, was $73.8 billion.

### Motor Vehicle Crashes—Drowsiness, Sleepiness, Falling Asleep at the Wheel:

The NHTSA estimates that approximately 100,000 police-reported crashes annually (about 1.5% of all crashes) involve drowsiness/fatigue as a principal causal factor (NHTSA, “Mission of Drowsy Driving Program”). At least 71,000 people are injured in fall-asleep crashes each year. A conservative estimate of related fatalities is 1,544 annually, or 4% of all traffic crash deaths. If applied to the 18-29 year-old driver group, drowsiness was culpable for at least 530 fatalities. Furthermore, according to a publication by the NSF, “Facts About Drowsy Driving,” drowsiness/fatigue likely plays a role in crashes blamed on other causes. About one million crashes annually—one-sixth of all crashes—are thought to be produced by driver inattention. Hartley, et al (1996), however, proposed that up to 25% of all US crashes, far more than estimated by the NHTSA or the NSF, are due to failures of attention on the part of the driver either owing to outright sleep or alertness lapses due to the onset of fatigue. Until recently there were no standardized criteria for determining driver sleepiness. Even with provision for records, proof is difficult because there is no test to confirm its presence as there is for intoxication (i.e., a “breathalyzer”). Nonetheless, some organizations have risen to the challenge. In a paper presented by the Pennsylvania Turnpike Commission (Hickey), project studies revealed that 57 percent of all run-off-the-road crashes on the turnpike from 1990 to 1995 were caused by drivers falling asleep. Similarly, the New York State Thruway Authority NEWS of Feb. 18, 1997, reported that fall-asleep fatal accidents on the Thruway system between 1991 and 1995 averaged 12 a year, or 35 percent of total deadly accidents on the system. In both of these road systems, and recently others, the approach has been to add “shoulder rumble strips” which, when rolled upon by tires, create noise and vibration in attempt to snap the driver back to alertness.

Economic Impact, Crashes Involving Drowsiness, Falling Asleep At The Wheel—Sleep disorders, often involved with traffic accidents, impinge greatly on national economics. The NHTSA estimates that drowsiness crashes represent $12.5 billion in monetary losses each year (NSF, Drowsy Driving).

Statistics from NSF's “Sleep In America,” polls, conducted in collaboration with The Gallup Organization, showed averages over years 1995, 1998, 1999, 2000, 2001, and 2002 as follows:

Practically half (49%) of adults in the U.S. had driven a car or other vehicle while drowsy within the past year, peaking in 1999 at 62%. The average for drivers 18-29 years old was an astounding 68%.

Disturbingly, more than one in five respondents (22%) admitted to having actually dozed off at the wheel during the past year. More appallingly, nearly one in three (29%) of drivers 18-29 years old had fallen asleep.

Of all adults surveyed, 1.65% claimed to have had an accident while driving at some time because they were too tired or they dozed off.

At the 2002 National Summit to Prevent Drowsy Driving, David Dinges, an expert on sleep and body clocks from the University of Pennsylvania School of Medicine, said dozens of claims had been made for devices that were installed in vehicles as “online monitors for drowsy driving.” The problem, Dinges said, is finding something that works reliably for nearly everybody, in nearly every situation. Also at the Summit, Mark Rosekind, an expert on measures to counteract fatigue, formerly with the National Aeronautics and Space Administration, agreed that no such device has been adequately tested for effectiveness and reliability. A major problem with most of the gadgets, he said, is how people defeat the purpose of them, sometimes unwittingly (Brody). Thus, the problem is similar to that of electro-mechanical alarm clocks, which are easily disabled. A more direct means for reducing the occurrence of accidents, by preventing the very tendency to drowse and enabling drivers to be in more vigilant control of their vehicles, would therefore be preferable.

The NSF emphasizes that sleep-related crashes are most common in young people, who tend to stay up late and sleep too little (Facts About Drowsy Driving). A bulletin from the State of North Carolina DOT advised that 55% of fall-asleep crashes involved people 25-years-old or younger in North Carolina.

The correlation between the disproportionately high representation of young adults having sleep problems and elevated incidence of young drivers involved in traffic accidents is, seemingly, more than mere coincidence. The connection is especially apparent regarding the difficulty in getting out of bed and the magnitude of accidents in morning traffic.

### Early Morning Driving, Including Youth Drowsy Driving

Multiple studies of police crash reports wherein the driver dozed at the wheel, by Pack et al.; Knipling, Wang; New York State GTSC Sleep Task Force, 1994; New York State Task Force on Drowsy Driving, 1996; Langlois et al.; Lavie et al., 1986; Mitler et al; and Home, Reyner; demonstrated that drivers 25 years of age or younger experienced accidents by falling asleep most frequently around dawn and during the morning rush. Similarly, in the NCSDR/NHTSA report, data and tables reveal that of all accidents involving 25 year old or younger drivers over the full 24 hour day, 25% occurred between 6:00 AM and 9:00 AM, in contrast with 16% as the approximate average for the combined other age groups.

A significant report to Congress on Jan. 19, 1993 by NHTSA (DOT HS 807 957), was provisioned with a graph of “Number of Crashes by Time of Crash and Age Group.” Between the hours of 6:00 AM and 9:00 AM in 1990, drivers of the 15-24 years old age group were involved in almost one fourth (23.8%) of the total accidents involving all ages (296,500), yet the 15-24 years old drivers represented only about one eighth (13%) of all licensed drivers.

The high accident occurrence around and after dawn is especially significant considering that the 6:00 AM to 9:00 AM time segment represents only 12.5% of the day. Comparable data for drivers 18-29 years old, a major statistical age group examined by the NSF, are similar. Presumably, a major portion of situations involved drivers in transit to work, the high crash frequency indicating that the drivers were still half-asleep from somnolence when starting out.

Further Economic Impact, Dawn and Early Daylight Hours—From the graph of DOT HS 807 957, the 1990 sum cost for all wrecks between 6:00 AM and 9:00 AM, including every age group, appears to have been $6.3 billion. In 2000, the cost of crashes for this fraction of the day was $29.5 billion.

### Adolescent School Performance

In addition to placing young people at high risk for automobile crashes, sleep problems may cause tardiness and even interfere with school attendance. According to the NSF 2000 survey more than one-third of the parents/guardians surveyed said adolescents are hard to get up (38%). Specifically, about one out of ten say their adolescents are “very difficult” to get up on a school day. In particular, it is arduous to wake up older teens, 17-18 years old. Moreover, NSF statistics show that one out of ten adolescents (10%) are late to school a few days per month or more due to oversleeping or being too tired. A study by the Center for Research on Child and Adolescent Mental Health Services in the Department of Psychiatry at University of California at San Diego concluded that some high school students suffer from DSPS.

The U.S. Congress is Concerned with the Wide Impingement of Sleep Problems

A 1993 congressional report (NComSDR) estimated that in 1990, sleep disorders and sleepiness cost the United States a minimum of $15.9 billion in direct expenses alone. This did not include the billions of dollars in indirect and related costs, such as those culpable for sleep-related tragedies, e.g., Exxon Valdez grounding, space shuttle Challenger disaster, and diminished productivity in the work place. Accordingly, as part of the National Institutes of Health Revitalization Act of 1993, the National Center on Sleep Disorders was founded.

Subsequently, the Occupational Safety and Health Administration (OSHA) brought to the attention of lawmakers that sleepiness, as a result of either untreated sleep disorders or simple sleep deprivation, was a causal factor in many chronic diseases as well as a growing number of vehicular and on-the-job injuries. Sleep deprivation was also recognized as a growing problem for high school students, the largest at-risk group for fall-asleep car crashes. In response, The Senate Committee on Appropriations, in October, 2001, passed a bill (S.1536) which included funding to enable the Center for Disease Control and Prevention (CDC) to establish and begin implementation of a 5-year sleep awareness action plan designed to develop public health programs regarding sleepiness and sleep disorders nationwide (Senate Report 107-84).

Furthermore, the first federal bill (H.R. 5543) addressing the issue of drowsy driving was introduced in the House of Representatives by Rep. Robert Andrews (D-NJ) on Feb. 27, 2003. It is known as the “Maggie's Law: National Drowsy Driving Act of 2002 (HR 968).” This intends to amend Title 23, United States Code, to provide incentives to states for generation of traffic safety programs to reduce crashes involving driver fatigue and sleep deficiency.

### Productivity

In addition to traffic accidents, sleep disorders, especially those involving inability to awaken with reasonable dispatch, are related to workplace problems. Lost productivity is a pervasive concern, and its major components include absenteeism, turnover, and accidents. In a study by Lavie (1981), productivity was seen to be lost and work satisfaction was worse for insomniacs. Johnson (1983) demonstrated than insomniac navy men were slower at work and had poorer career advancement than good-sleepers.

### Absenteeism

As publicized by the BLS, the median number of days away from work for all workers in all industries rose from 5 during 1994-98 to 6 in 1999 and 2000. In 2001, for all full-time workers, the Total Absence Rate equaled 3.6%, of which 2.5% was due to injury or illness, and 1.0% was due to “other reasons.”

Absenteeism is strongly associated with insomnia. Stoller demonstrated that people with insomnia have increased absenteeism, in addition to reduced productivity. Leigh, in a survey of 1308 workers, found that insomnia was the most predictable factor of absenteeism at work, evidenced by insomniacs having an average monthly sick absence rate 2.8 times that of the total group.

Generally, it can be presumed that insomnia will subsequently hinder getting out of bed at the scheduled time point. As mentioned previously, the NSF poll showed that 14% of adults confided that they were often late to work due to sleepiness. Moreover, some adults polled in 2000 said they had completely failed to go to work because of sleepiness occasionally or frequently. It can be inferred that afflicted workers, upon oversleeping, decide that they will do less damage to their employment record by being absent and taking an allotted “sick day” than by arriving tardy. Since accumulated tardiness and absenteeism are major reasons why adults are dismissed from their jobs, oversleeping and/or sluggishness in ascending to full alertness may not only reduce productivity, but can jeopardize the very survival of affected individuals.

Fiscal Impact of Insomnia—The cost for losses of productivity due to insomnia in the U.S., including that resulting in absenteeism, was estimated by Stoller to be 41.1 billions US$ in 1988. In 2003 dollars, this would be closer to $100 billion.

The survey conducted annually by CCH is not only the most definitive on absenteeism, but also the only study that measures costs associated with unscheduled absences. The Mean Rate of unscheduled absenteeism, for years 1994-2002, averaged 2.5, lower than the high of 2.9 in 1998. The 2002 survey results were released Oct. 16, 2002. Although the annual absenteeism rate remained relatively steady at 2.1%, the average cost per employee reached a new peak at $789, up from $755 in 2001 and surpassing the previous high of $757 from 1998. The average cost for 1995 through 2002 has been $669 per employee.

As an additional element of workplace efficiency, accidents in 2000, according to the National Safety Council (NSC), cost the nation $131.2 billion in decreased productivity and other expenses. If as little as 8% of that figure is SD related, then an annual cost of $10.5 billion would be ascribable to sleep deprivation.

Difficulty in making the transition from sleep to wakefulness can be caused by numerous SDs, including hypersomnia, DSPS, and sleep paralysis. Insomnia is probably the most common SD contributing to wakeup difficulties, but what is the root of the insomnia?

### Stress

Sleep dysfunction is also involved with stress, which apparently may be either a consequence or a contributing cause of workplace adversity, or both. The NMHA advises that stress is nearly the greatest of all problems in the workplace, following only family crisis. According to the 2002 CCH survey, among the reasons people called in sick at the last minute, stress was cited in 12 percent of occurrences, peaking in 2001 at 19% (tripled from 1994).

Stress is especially counterproductive to office workers, wherein long hours, and pressure to produce, along with unrealistic goals, are inflicting insomnia and even illness. Random telephone surveys of 2,511 U.S. workers were conducted by Integra, finding the following statistical averages:

Two-thirds of American workers (63%) affirmed workplace stress was a problem for them at least occasionally, and one out of every 10 workers (10%) said workplace stress was a major issue for them.

More than one in eight (11%) said they had called in sick sometime in the past year due to stress at work.

One in five (20%) had quit a job in response to stress (see the discussion of Turnover below).

A major determination by the Integra studies, that many office employees have called in sick because of stress at work, is inauspicious considering that absences have a great impact on national economy. The resultant absenteeism is a component of the above cited “other reasons” categorized by the BLS.

Moreover, according to the surveys, workplace stress has caused 32% of Americans to be unable to sleep.

Since insomnia has been clearly shown to be a factor of absenteeism, it can be concluded that stress often compounds tardiness and absenteeism, possibly impelling a continuous state of stress and precarious employment. Indeed, NSF poll respondents attested that stress interfered with sleep. Between 1995 and 1999, an average of 38% of adults emphasized that stress adversely affected their sleep. In turn, at least two-thirds (66%) of adults in the 2000 poll said that sleepiness made handling stress on the job harder.

From the preceding expositions, it is reasonable to expect that when such stress reaches a level sufficient that a worker is inclined to call in sick, and stress is coincident with lack of sleep or oversleeping which disposes him or her to avoid going to work, the employee probably will be absent that day.

Fiscal Impact of Stress—The estimated annual cost of job stress to U.S. industry, according to the AIS, is a staggering $300 billion. This figure includes the costs of diminished productivity; absenteeism (between 225 and 275 million workdays lost yearly in the U.S.); and employee turnover.

### Turnover

Besides being major workplace problems in their own right, both absenteeism and stress can be factors of turnover. The AIS advises that the portion of national job turnover due to stress is 40%.

Annual surveys conducted by BNA demonstrate that the annual nationwide median turnover rate has risen strongly from 10.8% in 1996 to more than 15% in 2000. Worse, the first release of the new Job Openings and Labor Turnover Survey (JOLTS) from the BLS estimated a far higher rate, 39%, for the 12 months from May 2001 to May, 2002.

From the Integra revelation concerning quits, and the NSF data regarding job changes, it is evident that when both elevated stress and extensive sleep deficiency are experienced, turnover is eminent.

Notably, changing jobs is itself not only stressful, but perhaps traumatic. In the classic scale by Holmes and Rahe, job change ranks among the eighteen most stressful of possible life events.

Beyond question, sleep dysfunction and personal problems at the individual worker level can collectively affect employment at the macroeconomic level. The converse sequence also can be seen to occur. It is well known that mass layoffs, for instance, escalate workers' feelings of insecurity (Norris). Plainly, such apprehensions can only add to stress, and thus exacerbate sleep dysfunction by promoting recurrent patterns of insomnia and difficulty in awakening, perhaps actually precipitating resignations, dismissals, and turnover.

Fiscal Impact of Turnover—Estimates regarding the actual costs of turnover range from 33 percent of the employee's base salary to as high as 250 percent of that salary (Cheney).

### Depression:

For more than one sleep disorder, substantial evidence suggests that depression may play a significant role. Aldrich discovered that up to ⅔ of patients with DSPS are depressed at the time of evaluation or have been diagnosed with depression. That analysis also revealed that unemployed or disabled persons are at risk for DSPS due to lack of a regular work schedule. It can be alleged that, in turn, the DSPS causes trouble in obtaining and keeping employment. Thereby, the sufferer may become caught in a behavioral loop, then fall into depression.

Results from the NSF's 2001 poll revealed that adults who are most likely to experience a sleep problem include those who suffer from clinical depression (83%). That study also found that sleepiness, interfering with one's daily activities at least a few days per month, is more common among those experiencing periods of depression (58% vs. 32%-48%) than among their counterparts.

In a study by J. C. Ware and J. Morewitz, of 1,061,396 patients who visited physicians for insomnia as the primary reason for the visit, 31.7% were diagnosed as having depression. Numerous other studies have documented the relationship between depression and insomnia (Bixler, et al; Mellinger, et al; Frisoni, et al; Liljenberg, et al). Affirmatively, the Surgeon General's Report on Mental Health explains that, when accompanied by other symptoms, insomnia can be a classic sign in diagnosing depression.

The NIMH advises that major depression is the leading cause of disability in the U.S. and worldwide. And, according to a recent large-scale study by Wells, et al, depression results in more days in bed than many other ailments. Moreover, depression ranks among the top three workplace problems, subordinate only to family crisis and stress (NMHA). Interestingly, a separately classified disease, Chronic Fatigue Syndrome, in outward appearance resembles a combination of most of the symptoms of depression.

Fiscal Impact of Depression: As for macroeconomic detriment, the definitive study was by Greenberg, et al. This investigation revealed that as many as ten percent of all adults experience clinical depression each year, with an estimated annual cost to American businesses of $43.7 billion including absenteeism ($11.7B), lost productivity ($12.1B), and the direct costs of treatment and rehabilitation ($12.4B). If as little as 15% of these costs are ascribable to SDs, that portion is culpable for $6.6 billion.

In summarizing the relationship between slumber and productivity, a complex interplay can be seen between sleep dysfunction, stress, depression, and troubles in employment stability.

### Chronic Fatigue Syndrome:

A common simple definition for Chronic Fatigue Syndrome (CFS) is: A condition of prolonged and severe tiredness or weariness that is not relieved by rest and is not directly caused by other maladies.

The CDC estimates that CFS affects as many as half a million persons in the US.

As explored by Norma C. Ware, Ph.D., people with CFS have a 50% probability of losing their jobs as a direct result of their condition. Their symptoms, such as persistent fatigue, headaches, fever, and depression, make getting to work in the morning a challenge because joint pains may prohibit those with CFS from grasping a steering wheel, or they might fall asleep en route.

The US Congress has acknowledged that CFS remains an enigma to the international medical community and no proven effective treatment exists, while the condition causes combined symptoms of depression such as fatigue, malaise, and sleep disorders. Accordingly, the 103D Congress passed House Joint Resolution 264 on Sep. 22, 1993, designating the month of March 1994 as “Chronic Fatigue Syndrome Awareness Month.”

### Customary Approaches for Assisting the Transition from Sleep to Waking

### Clocks Featuring Signaling Devices

Industrial science up to the present has approached the transition from sleep to waking predominately by external mechanical and electro-mechanical timing devices, evolution being represented by a surfeit of contrivances, mainly consisting of combinations with radios, and lately microcontrollers. The ubiquitous alarm clock allows the user to prearrange an impetus to be delivered upon himself at a certain near-future time point. At bedtime, the device is adjusted to suddenly commence a stimulus after elapse of a chosen number of hours.

Prearranged stimulus by clock devices featuring signaling or alarms has become an ingrained constituent of modern civilization. However, in their main service of awakening people from sleep, these mechanisms are all characterized by the weakness of having to impart a stimulus from a position which is external to the user's body. The individual must then try to exit the bed, before physiologic changes are complete. Moreover, a ruinous tendency of the sleeper to disable the alarm often causes further gain toward invigoration to become impossible. One variation is the automated “wake-up call” from hotel desks. These are ever more unappealing and ineffective for travelers, and like an alarm clock, the purpose of the system can be defeated, in this case by simply hanging up the telephone. With either arrangement, even if the individual does return to the conscious state, onset of alertness and rising motor potential are very gradual at best, and more often are quite slow.

### Caffeine-Containing Beverages and Solid Oral Dosage Forms

A related facet of transition from sleep to waking is the customary boosting of alertness pursuant to wakeup with beverages which contain caffeine. Chemical agent ingestion by imbibition of stimulant beverages is a rudimentary means of arousal by internal physiologic control. An expedient yet equally inferior derivative is the swallowing of caffeine-containing tablets, capsules, etc., normally chased by a neutral beverage. With either method, actual change of state of consciousness must already have occurred before the individual can conceivably imbibe anything. Therefore, such conventions can only serve to supplement some previously attained level of wakefulness. Another disadvantage of chemical agent ingestion is related to the habitual haste of young adults in getting out the door and onto the road. Because of this hurry, they frequently skip a cup of coffee or other means of increasing alertness before operating a vehicle. Some intend to stop for coffee along the way, but nonetheless must first expose themselves to danger by driving in a semi-alert condition. An additional inconvenience of unimproved stimulant ingestion is that the setup of beverage percolators for preparation of caffeinated drinks is laborious, time consuming, and can be especially messy. Also, in recent years, there have been cases of real scalding by hot liquids, not only of the mouth, but to other body areas, from spillage.

### Counteraction of Sedative Hangover

Herewith, it may be appropriate to mention yet another area of scientific research which is secondarily related to the field and background of the present invention. For decades, insomnia has been treated with pharmaceutical tranquilizers. Unfortunately, the preponderance of these agents, including many benzodiazepines, e.g.—diazepam (Valium), and lorazepam (Ativan), are characterized by long half-lives, causing the patient to experience a “hangover” after whatever success they may get at sleeping. The hangover effects during the next day may include impairment of psychomotor performance (Beets), confusion and poor concentration (Baldessarini), as well as apathy (Lader). Some authors (Skegg, Richards) have drawn attention to the probable contribution of benzodiazepines to traffic accidents.

Flumazenil, or ethyl 8-fluoro-5,6-dihydro-5-methyl-6-oxo-4H-imidazo(1,5a)(1,4)benzodiazepine-3-carboxylate, is a pharmaceutical agent manufactured by Roche under the trade name Romazicon, and has been in use for several years as an antidote to benzodiazepine anesthesia and overdose. According to the monograph, flumazenil is an imidazobenzodiazepine derivative having ability to obstruct the actions of other benzodiazepines. Administration of the formulation is by intravenous route only (Romazicon). Pursuant to injection, the drug leaves individuals alert and able to perform at normal levels. Unfortunately, development of an approved orally-administered formulation will take years. Furthermore, it is not a stimulant and is ineffective in countering sleep deprivation (Wesensten). Obviously, its value would likewise be negligible as a primary treatment for those sleep disorders whose symptoms include trouble in making the transition from sleep to wakefulness.

Reportedly, research has been conducted by the U.S. Army for a field-deployable sleep management system to maximize individual and unit performance during continuous military operations. The assemblage will include a sleep-inducing, rapid-reawakening drug combination. This arrangement incorporates the agent flumazenil. Given upon awakening, it is intended to reverse the hangover effects of sleeping tablets which constitute the other side of the combination. The projection supposes that an orally administrable form of flumazenil will eventually be available (Belenky).

There is no prior art on record regarding any drug combination with agents having reverse actions. And, at present, there is certainly nothing describing any oral form of flumazenil. Therefore, the scheme as disclosed will have serious weaknesses. In the first place, flumazenil can be administered only by parenteral means, and consequently the drug cannot be configured for delayed release and taken simultaneously with the sedative at bedtime. Also, as flumazenil is not actually a stimulant, the preparation will not be able to positively promote transition from sleep to wakefulness. Conceivably, the agent could be injected while the individual was still unconscious with hope that compunction of the needle would effect arousal, but the procedure would be susceptible to a horde of adverse events. More realistically, the recipient would first have to be awakened to receive the injection, drawing out the start, and requiring more time for initiation of action, thus compromising efficiency.

It has now been discovered that a superior means for organizing the wakeup process is through administration of a refined pharmaceutical dosage form at bedtime the night before. The device introduced herewith renews technical progress in aiding transition from sleep to waking, principally by awakening a patient following a specifically delimited time span via prearranged internal physiologic control.

Considering the numerous health problems above and obvious need for an improved means for arousing individuals having troubles with making the transition from sleep to wakefulness, it is a wonder that the idea of the new invention has not been conceived previously. One plausible reason may stem from the notion that stimulating agents should not be ingested late in the day. This maxim could be so inviolable, even for scientists, that it precludes the very ability to imagine a dosage form allowing no immediate release of stimulant which could be taken just before bedtime. What is more, some of the logic involved in the concept of the device verges on a paradox loop. This occurs in the reality that although transition to wakefulness is a major object which the formulation aims to effect, the recipient must already be awake in order for the oral dosage form to be administered. And, conversely, since the preparation cannot be administered without the recipient being awake, accomplishment of pharmaceutical wakeup seemingly cannot be realized. Normally, issues such as this tend to immediately conflict with common sense, and in the minds of most people, the reaction is to dismiss further train of thought.

### Description of the Prior Art

The new invention, as a single dosage pharmaceutical means for both assisting onset of sleep and later inducing wakeup, and in its appurtenant beneficial aspects, is unprecedented. For purposes of comparison and prelude to the Specification, however, some existing drug release methods merit retrospection.

Historically, the Persian physician Rhazes, ca. 865-925 A.D., is credited with invention of the coated pill. Traditionally, coatings have been used for some simple prospect of postponement, whether for taste masking, digestive tract site targeting, or other purposes.

As for modern configurations, nearly all coated solid forms are for extended release of active components over a span of time, by either diffusion or erosion. These delivery types are completely familiar to those skilled in the art. In all such forms, there is generally some release of the active agent immediately. Usually, after this initial discharge, controlled delivery formulas rely upon sustained or “constant” release so as to maintain a consistent therapeutic blood level to treat specific diseases. Refer to Seitz, J. A., et al.

Advancing beyond the group of simple sustained release formulations for delivery, however, is the less common category referred to as “delayed-release,” wherein delivery of the main portion of drug is withheld for some period of time. A good means for accomplishing this is by a dialysis membrane, normally created by the coating of an active compound-containing core with an appropriate film-forming material such as a lacquer. Usually, preparation is by spraying solutions into fluidized or spouting beds which provide concurrent drying of the substrates being coated. Once the routine is finished, the film cures into a thin yet tough layer which most commonly is perforate with relatively large pores and across which, in aqueous media, a pressure gradient is developed. The core materials constitute the heavier concentration in the gradient, thus drawing aqueous gastro-intestinal media inward by osmosis. When such a formulation is taken, the release is at first controlled by outward permeation of active compound through the pores. Ordinarily, the diffusion is accompanied by progressive erosion of the coating layer, with ultimate disintegration releasing the remaining quantity of active agent. In somewhat more extraordinary forms, the coating ruptures to suddenly deliver active contents. The principle and technique for preparation of bursting membranes are well explained in U.S. Pat. No. 3,952,741 (Baker), classified as a dispenser.

Henceforth, and in the above discussion of coatings, the term “lag” always refers to that overall span of time measurable from the point of administration of a pharmaceutical dosage form to intended response. The term “delay” always refers to release and/or delivery of the medicinal agent, and specifically to that portion of the lag time which is most practically programmable by pharmaceutical technique.

The earliest patent application describing a bursting osmotic membrane is U.S. Pat. No. 2,478,182 (Consolazio, for the US Navy, Aug. 9, 1949). The inventor discloses sodium chloride tablets, dip-coated in a polymer solution. The resulting delivery mechanism operates by diffusion and then bursting of the tiny cellular compartments from inner pressure. Although ingenious, this system cannot delay release for more than one hour due to the lack of continuity of the membrane and absence of significant elasticity.

U.S. Pat. No. 3,247,066 (Apr. 19, 1966) discloses medicinal beads that likewise release drug by a bursting mechanism. A principal aim is controlled release of the active compound regardless of the pH of the various body fluids. The beads each consist of a core of water-swellable colloid containing an active agent which is fully surrounded by a thin membrane formed by a polymeric coating. When ingested, water diffuses inwardly through the coating causing the core to swell. Ultimately, the thin polymer layer ruptures, thereby releasing the drug. Although some minimal delay times can be achieved by this system, it has inherent deficiencies. For instance, the membrane is supplemented by only a primitive plasticizer or no plasticizer whatsoever, thus there is very little expansion possible antecedent to bursting, and the preparation is unable to delay release for more than a few hours. Both the membrane and the colloid core composition are responsible for other operational shortcomings. One disadvantage is seen following elapse of the delay, where the rate of release becomes increasingly slower as, by adjusting the coating solution or technique, the designated interval is lengthened. Such release behavior is visually recognizable by inspection of graphics which plot percentage of agent delivered on the vertical axis against time on the horizontal axis. Plots for membrane-coated core formulations of this type generally have a characteristic “S” (sigmoid) appearance, where the top curve represents post-delay prolonged release. In preparation, when attempt is made to shorten the absorption time which follows rupture, the result is excessive lengthening of the delay interval and a progressively more unpredictable bursting point, thereby causing erratic, and thus inadequate, performance. If a compensational effort is made to contract the disproportionate delay interval, the corollary is not only an unacceptably prolonged post-rupture release, but unwanted leakage. Indeed, the data tables of U.S. Pat. No. 3,247,066, especially in Example 6, clearly show that the dosage forms leak more than 50% of the core contents before the time of bursting. And, although no graphic analysis was provided by the inventor, a plot from the data table of Example 9 has now been prepared, and is included as FIG. 4 in the drawings accompanying the current application. In the given example, attempt is made to vary the delay interval by changing thickness of the mediocre membrane, resulting in directly proportional changes of the post-delay prolonged delivery and inversely proportional changes in the degree of precocious release. Leakage, typical of virtually all known rupturable membrane prior art, is illustrated by the lower curve on the sigmoid shape of the profile. Commonly in such preparations, leaching of drug has an almost immediate onset with the rate accelerating toward the end of the planned delay interval. In the subject prior patent, the weak membrane is again culpable, of allowing diffusion ahead of complete rupture. The behavior profile here is further explainable in that, when rupture occurs, the colloid is actually released as a cohesive aggregate and must undergo further dissolution before the active agent can fully disperse. This last deficiency is the source of a drawback having direct influence on patient response, the undesirable trait being availability of only a minor portion of the dosage for absorption initially, and continued uptake of the remainder over some indefinite time period.

U.S. Pat. No. 5,496,561 (Okada), is somewhat similar to U.S. Pat. No. 3,247,066, a pair of differences being the addition of silicone oil to the membrane mixture and cores which are created by buildup onto starter seeds rather than as colloids. This preparation likewise exhibits a very typical sigmoid-curve release profile (see FIG. 5) wherein rate of release becomes increasingly slower as attempt is made to program the delay for a longer interval.

European patent EP 1074249 and World patent WO 00/74655 are further derivations of predecessors such as U.S. Pat. No. 3,247,066, the major difference being addition of a preliminary release. The inventors of EP 1074249 use the term “fragment” rather than burst or rupture. in all examples the first release is sustained for several hours, with at least one situation showing a 2-plus hours delayed initial release followed by such sustain, and in all examples the final delivery is deliberately configured for extended release. Although the use of more than one agent within a single preparation is claimed, the provision is only for the final, extended release.

Although the inventor of WO 00/74655 claims that different agents may be included, the implication is that all agents in any one preparation are for the same purpose, or for treatment of the same medical condition.

For either the European or World patent, as in the other related prior art, when attempt is made to postpone the final release for more than a few hours, a sigmoid release profile is exhibited wherein rate of release becomes increasingly slower. Also, leakage becomes evident. In neither patent is any claim whatsoever made wherein the agents have opposite or reversive actions. Furthermore, none of the methods of treatment involve SDs, wakeup of a sleeping individual, or any other aspect of the sleep cycle.

All of the prior art pharmaceuticals above share similar disadvantages. Specifically, none are able to delay release for more than a few hours without forcing the final release to become sustained. The other common disadvantage, having a mutually detrimental interdependency with the previous, is lack of strict inhibition of premature release.

### SUMMARY OF THE INVENTION

This invention introduces an oral pharmaceutical sleep-management system relating to states of consciousness and effecting transitions between them, the initial transition involving onset of drowsiness, a subsequent phase corresponding to a nominal interval of sleep, and a conclusional transition reverting to wakefulness. The innovation optimizes a patient's sleep cycle by pharmaceutically assisting in both falling asleep and subsequent timely awakening. Further benefit is realized through improved alertness and vigor during the early hours following wakeup.

### OBJECTS OF THE INVENTION

The foremost object of this invention is to introduce a system which therapeutically regulates sleep by means of a new pharmaceutical dosage form. The initial tactical object is to establish a physical mode in the patient which is compatible with sleep, principally just after administration of the dosage form. The conclusive tactical object of the design is to issue an internal arousal impetus, but only in conformance with a scheduled timepoint. The ultimate strategic object is punctual wakeup of a sleeping individual by the previously administered medicine, following a nominal session of sleep.

The preparation must therefore, as a requisite, have ability to withhold release with fine precision until the intended chronological point of arousal agent delivery. A converse requisite is that the sleeping patient is not disturbed by precocious stimulation, i.e.—nervous system provocation commencing other than just before elapse of a specific measure of hours.

Accordingly, another important object is innately fashioned precaution that active agent will not prematurely leak from the dosage form.

Yet another object is provision for preparations in which active agent delivery is independent of the effects of any specific gastro-intestinal environment, thus enabling consistent and predictable chronology of action.

In designing the preparation, a concomitant goal was to arrange the calmative component so that overlap would be prevented in the schedule of its duration relative to the beginning of reversional action of the arousal agent. Constantly, the tonic, relaxant, or calmative agent is preferably characterized by rapid or moderate onset and contracted longevity. Indeed, realization for the entire concept was partly due to introduction over recent years of short-acting tranquilizers whose qualities exclude practically all side effects. Most favorably, suitable agents should promote natural sleep cycles. It is an intention of this innovation to employ calmatives succinctly, as well-integrated complements, thus accentuating judicious circumscription to a novel medical formulation.

Still another object is delineation of assessment criteria, for successful performance, which should be met by the design. As a minimally acceptable result, the pharmaceutical must promote awakening on a reasonable timely basis when supplemented by conventional alarm clocks and related devices. And optimally, given a sufficiently therapeutic dose of arousal agent, the preparation should induce enough neural activity to push a patient across the threshold of consciousness and into the waking state, fully unassisted by any other source of stimulus.

The ultimate object is to provide an oral medication which is therapeutic for a considerable range of neurological conditions, including those involving impaired alertness and deficient vitality pursuant to wakeup.

Further requisites are set forth in the Detailed Description.

### General Advantages

The intrinsically synergistic advantages and benefits of the innovation engender a great step forward in the available means for dynamically initiating a new day.

A comprehensive principal advantage, attributable to the pharmaceutical character of the design, is that arousal provocation cannot be arbitrarily or spontaneously defeated by sleepers in their disabling of the stimulus, as too commonly does happen with signals from mechanical and electro-mechanical devices such as alarm clocks. It is quite impossible for a patient to annul the internal stimulation evoked by the device, and the insistence to become energetically active cannot be eluded.

Also, in stark contrast with unimproved chemical delivery by beverages or simple solid oral forms, the pharmaceutical agency is able to initiate physiologic changes as prelude to and proximal cause of wakeup, rather than in attempt to increase alertness after the user has already arisen. And, since provision for early alertness is arranged for on the preceding evening, the inclination of young drivers to omit increasing their alertness with coffee or other means before beginning their drive is inconsequential. Furthermore, because the new device reduces or eliminates necessity for the patient to imbibe such stimulant drinks while still torpid, accidents with messy beverage equipment and scalding are avoided.

Since most adults and older teens experience considerable trouble getting up out of bed in the morning, it is deemed that the medication will be exceedingly helpful to individuals from these age groups. The anticipated gains for students include better punctuality and attendance. The expected rewards for adults encompass improved workplace productivity and employment surety. When the workplace improvements are realized on a company level, the lowered frequency of tardiness and absenteeism, with correspondingly diminished turnover, can increase profits for businesses. If reduced turnover is realized on a broad scale, the utility could extend to moderation of macroeconomic unemployment.

As a benefit of residual blood levels of arousal agent, the usefulness of the invention embraces assistance of alertness for a spectrum of early waking activities. Daybreak and morning drivers may be able to reduce the probability of their being involved in motor vehicle accidents, attributable to the action of remaining stimulant. Regrettably, devices that are installed in cars and trucks and intended to prevent a driver from having a mishap exhibit the same weakness as do alarm clocks, in that they can be disabled. Also, rumble strips, built on roadway systems in order to reduce run-off-the-road crashes, can only issue a warning stimulus in drift-onto-shoulder situations. Thus, they are ineffective in preventing drift-into-other-lane impacts, as well as straight-line collisions, such as rear-end and crossroad wrecks. Moreover, rumble strips can easily fail if the driver is severely drowsy or falls completely asleep. The new pharmaceutical is intrinsically superior to such gadgets and road bumps in that once within the user's system, the remnant arousal agent will continue to assert action until the natural process of removal runs its course—over several hours. Meanwhile, the vehicle operator is incapable of switching off the internal physiologic stimulus. Thus, the importunity to remain alert will not relent before allowing sufficient time for an early drive of considerable duration at reduced-risk. Above all, the new formulation has fundamental advantage over external alarm provisions in that it is more direct, in its minimization or elimination of the very tendency to drowse, and imparting of sufficient vitality to drivers for them to be in vigilant control of their vehicles. If implementation is realized on a broad scale, the innovation could reduce traffic accident statistics.

The scope of the design further branches to treatment for sleep disorders and other syndromes, wherein difficulty making the transition from sleep to wakefulness at a reasonably scheduled time point may be even greater than experienced by the general population. This encompasses neurological conditions wherein symptoms include sequences or repeating patterns of sleep inadequacy accompanied by difficulty in awakening. As examples, Delayed Sleep Phase Syndrome (DSPS) and Chronic Fatigue Syndrome (CFS), in hindering punctual arrival or even attendance at work, both cause trouble in holding employment, and DSPS may impede actually obtaining employment as well. In turn, losing a job and difficulty in getting a job can certainly trigger or aggravate insomnia for susceptible individuals, thus instigating sleep inadequacy, and likely contributing to other disorders. The invention can interrupt such negative sequences, by improving punctuality, and reinforcing employment surety. Other SDs, including those which display insomnia as a symptom as well as those which may not, can also benefit from the new formulation.

At this point in time, there is no direct pharmaceutical treatment available for stress. Rather than just attempting to treat symptoms, such as anxiety, the invention deals with the very source of stress, namely, inefficiency and unnecessary adversity in striving to attain employment success and stability.

Orthodox therapy for depression consists primarily of administration of antidepressants, thus some aspects of mental well-being have not been addressed. In attending elements of positive self-concept, the new medication is pioneering.

Moreover, there is no proven effective treatment for Chronic Fatigue Syndrome. Pursuant to wakeup, residual arousal agent from the formulation reduces probability that CFS sufferers will fall asleep while in transit to work and fortifies their employment surety.

And, since depression has been shown to be associated with certain SDs, the innovation can further contribute to dissipation of vicious circles which encompass various combinations involving SDs, depression, and perhaps CFS as well.

### Specific Advantages Over Prior Patents

The only prior art patent applications equitably comparable to this novel invention are those which involve pharmaceutical delayed release for general, non-specific purposes.

One advantage of the new preparation over U.S. Pat. No. 3,247,066 is that it avoids usage of outdated, primitive plasticizers such as castor oil in the membrane ingredients. By replacement with advanced chemical-type plasticizers having strong solvent properties, there is provision for exceptional film continuity as well as increased elasticity. Concordantly, in conjunction with other materials upgrading, the membrane is capable of ample expansion before bursting, and the preparation is able to delay release for eight hours or more. Concomitantly, due to improved core composition, when rupture finally occurs, the contents are actually released as a non-cohesive solute and the active agent is in a state of superb availability. This last virtue is the source of another important advantage, which is a release pattern that implements prompt uptake of a large portion of the dosage and completion of maximal absorption over a minimal time period. And, a perfecting advantage of the new system is that active contents are not prematurely leaked.

As mentioned in the Background, practically all prior osmotic oral dosage devices intend to produce a sustained or controlled release profile. In vivid contrast, the present innovation, by nature of its very purpose and required performance, achieves a release profile which is essentially the antithesis; nothing is allowed to escape until after the designated interval of delay, then all of the active agent is unbound at once. The graphic representation of release by the new formulation is not sigmoid, but rather a steep linear profile. The strict delayed release design according to the invention is especially circumspect in this regard, since buildup of chemical tolerance, which stimulant agents are notoriously prone toward, is inherently avoided.

As a result of careful configuration and tight quality control for both core and membrane, the aforementioned tendency for progress of delivery to be ever slower as duration of the lag increases, is virtually eliminated. This difference constitutes a great refinement, for the purposes of the current design, over prior art such as U.S. Pat. No. 3,247,066 and U.S. Pat. No. 5,496,561. Furthermore, the new formulation provides for incorporation within a single dosage form of agents which are not only different, but opposite in their actions.

Moreover, a transcendent advantage is that active contents are not prematurely leaked by diffusion or other avenues. In concurrence with its ability to deliver 100% of the final active agent within a brief time period, the present invention is superior to the prior art.

### BRIEF DESCRIPTION OF THE DRAWINGS

FIG. 1 is a graph showing the results of dissolution and delayed release tests conducted for Example 1.

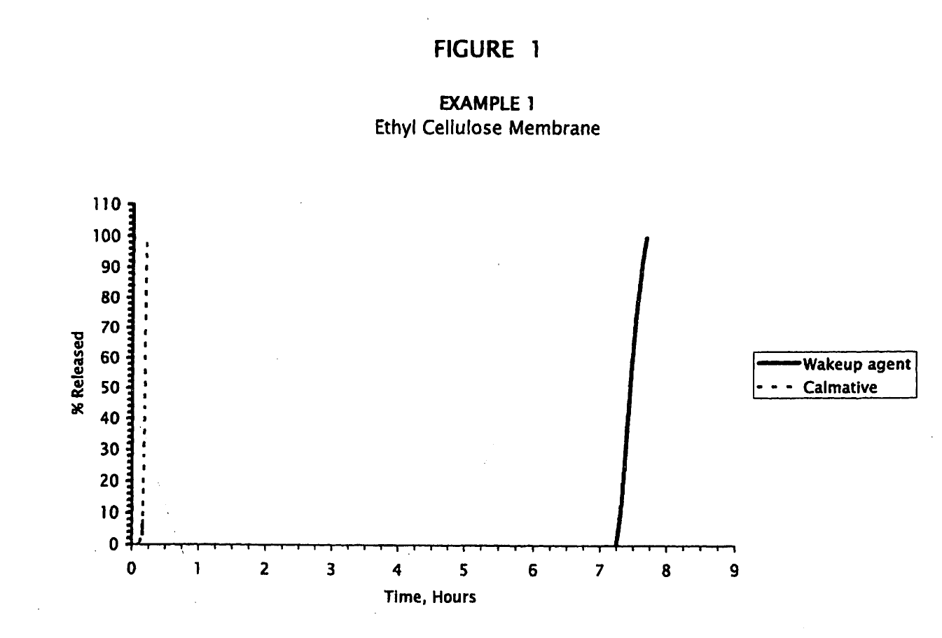

FIG. 2 is a graph showing the results of dissolution and delayed release tests carried out in Example 2.

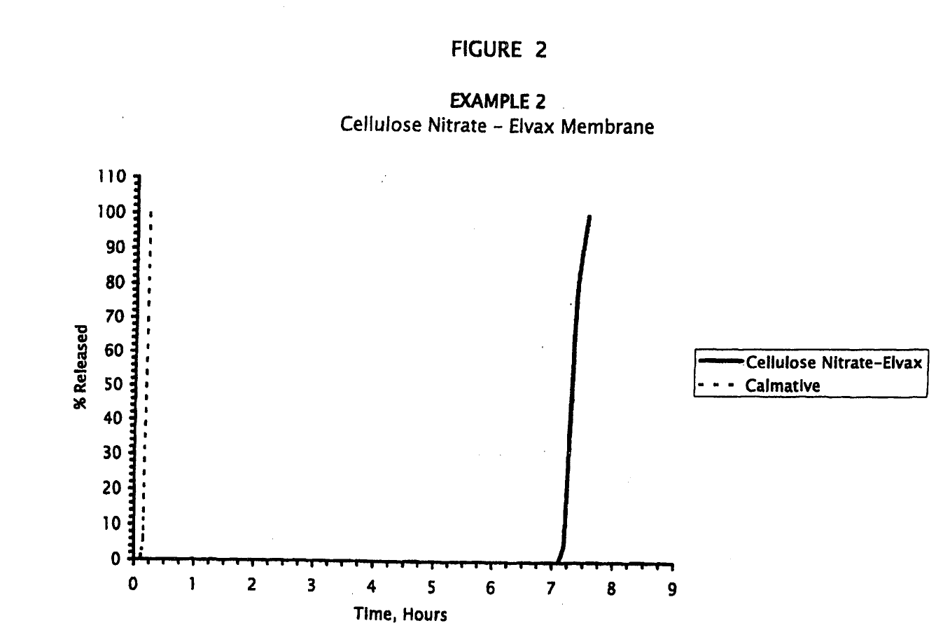

FIG. 3 is a graph showing the results of dissolution and delayed release tests from Example 3.

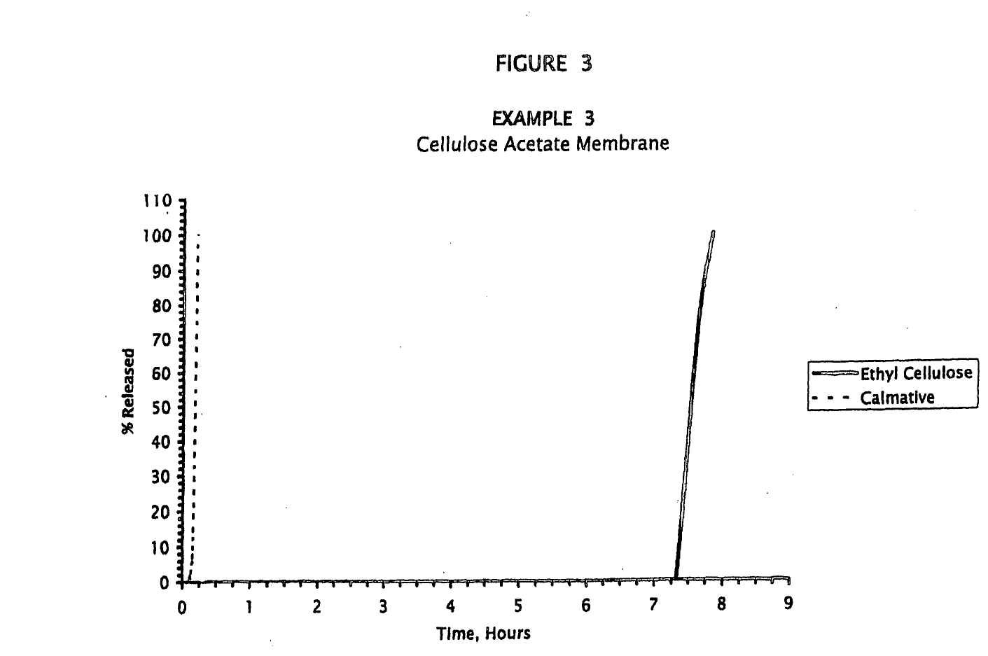

FIG. 4 is a graph showing the results of release tests from the ninth example of U.S. Pat. No. 3,247,066.

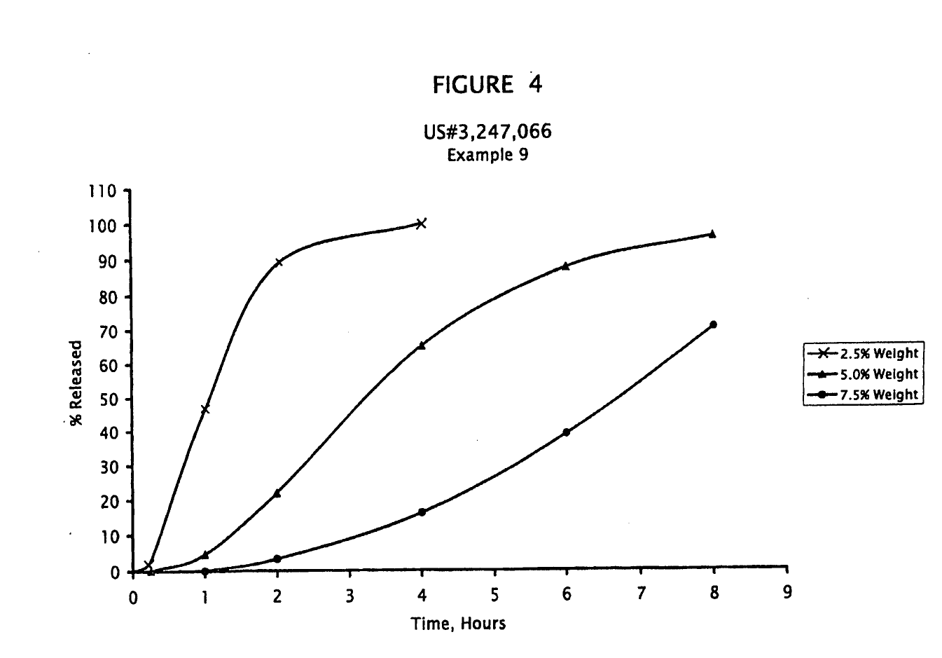

FIG. 5 is a graph showing the results of dissolution from the first test example of U.S. Pat. No. 5,496,561.

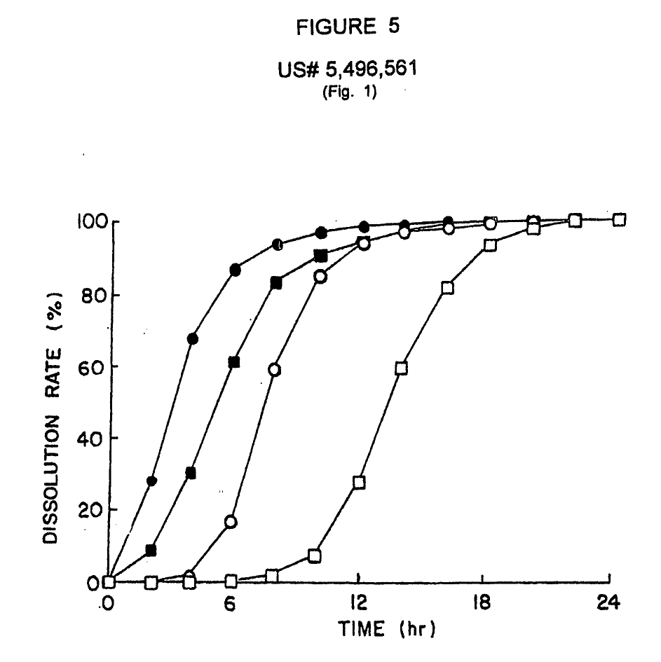

### DETAILED DISCUSSION

### Organizational Overview

For the preferred embodiment, a unit dosage is organized into an outer component group and an inner subsystem, all constituents being integral to the unitary device. The outer and inner areas have opposite functions, yet serve in mutual reciprocity to a common purpose, each generally carrying at least one distinct drug compound. Normally, upon administration, an initial tonic, relaxant, or calmative agent is promptly delivered from the outer component group and acts to promote onset of drowsiness. Following quite later, near the end of the patient's sleep period, a final agent from the inner subsystem is released which issues an internal arousal stimulus. In austere contrast to prior pharmaceutical arrangements, the initially and conclusionally released active agents are not only outright dissimilar as to chemistry, but the final agent is actually opposite in effects generated and reversive to conditions established by the previous active component. Such an association between pharmaceutical agents within a single dosage form is unprecedented.

### Opposite Actions

For most pharmaceutical agents in the “Product Classification Index” of the Physicians' Desk Reference, it is plain that there are no compounds known which are reversive. Where opposite-acting agents do exist, the possible combinations are nearly always absurd (e.g., antidiarrheals followed by cathartics; anabolics-catabolics) if not positively destructive (e.g.—antipyretics-pyrexials; anticonvulsants-epileptogenics; antidotes-poisons; anticancers-carcinogens), and thus offer negative utility.

Furthermore, for those few pairings which would not be either ridiculous or utterly harmful, (plausibly, hemostatics and anticoagulants; or antidiuretics and diuretics, etc.), and wherein delayed release would present no benefit, greater flexibility of control would be retained by administering the second drug separately. Therefore, such combinations would lack viable utility.

This general favorability of control by separate administration seems to apply to most pharmaceutically treatable medical conditions. A distinct exception is observed, however, in dysfunction of the sleep cycle. The primal truth here is that, while the recipient is unconscious, an active agent, sequential to a previous, reversive, or otherwise, cannot be administered by oral route, and cannot be self-administered by any route whatsoever. So, the issue in this situation goes beyond convenience vs. inconvenience or flexibility vs. forfeited flexibility, and becomes a matter of practicability vs. insuperability. Regarding the sleep cycle, therefore, the advantage seen elsewhere, of flexibility of control by separate administration, does not exist.

In concert with the major objects of this invention, a desired physiological effect to be attained by pharmaceutical administration is expedited transition from sleep to wakefulness. But, because the patient would still be asleep preceding the desired waking, the only way an oral arousal medication could be taken is in a delayed release formulation before actually falling asleep. And, since facilitation of transition from wakefulness to sleep is the other basic means of benefitting the sleep cycle, within these circumstances a combination of opposite acting agents, namely calmative with delayed-release stimulant, is not only entirely logical but unique in utility. Furthermore, upon observation that all other means of wakeup are easily disabled, the premium of such a pairing becomes apparent.

### Basic Structure

The most easily understood facet of the structural organization is the outer component group. Generally, this consists of a sleep-compatible layer, optionally covered by an outermost protective or beauty coat. This is fashioned similarly to most basic coatings, but may contain a calmative or tonic set for prompt release. Alternatively, the layer may carry some other complementary substance and no active agent, or may simply be a protective coating.

For the presently favored embodiment, provision for a certain interval of delay, as well as issuance of the internal arousal stimulus is accomplished by the inner subsystem. The subsystem incorporates at least one subunit. Ordinarily, this involves a core-coat configuration. The subunit structure is based upon a core which includes one or more pharmaceutically active agent with appropriate excipients, completely encased in an inert polymeric coating layer. The core may include any appropriate conventional excipients known to the art, including gas-generating substances such as, but not restricted to, sodium bicarbonate and calcium carbonate coupled with one or more mild acids such as citric acid and sodium dihydrogen phosphate. The coating constitutes an osmotic semi-permeable membrane, which is impenetrable by the drug, and although water-insoluble, is permeable to influx of water. This arrangement has been selected for its excellent ability to meet the objectives of independence from specific gastro-intestinal environments and precise delay without premature leakage.

### Operation

The basis of delayed-release operation for this embodiment is osmotic absorption of water over time through the semipermeable membrane. When a dosage unit is introduced to the gastro-intestinal (g.i.) tract, water vapor is drawn inwardly through the membrane layer of the subunit from the exterior environment. As the hydrophilic core material transforms into an osmotic solute, more water is imbibed into the subunit due to the osmotic gradient across the membrane. Time is consumed while the volume increases and an inner force is generated which distends and stresses the constitution of the polymeric layer. This process goes on until an area of the membrane encounters its moment of maximal elongation. At this point, structural integrity fails, marking the end of the delay phase. The bulb ruptures from inner pressure, creating large fissures, and the components of the core are positively and completely discharged to the g.i. tract. Such swelling will have advanced over a precalculated span of time, therefore completing coordination of a precise delay upon release. In application, stimulus and wakeup response are initiated upon absorption, and conclusion of the sleep interval is imminent. Preferably, after ingestion of the dosage form, is progressively expanded by internal pressure in synchronous correlation with residence in the gastrointestinal tract until the membrane bursts, thereby accomplishing delayed release of the wakeup agent.

It should be recognized, however, that the above described basis of operation is only the presently preferred means, and that delay of release of the final active compound may be effected by any permutation of the means known to the pharmaceutical art including, but not limited to, coated medicinal forms with selections of gastro-soluble, gastro-resistant and entero-soluble protecting layers; coated medicinal forms with a porous matrix and provided with a thin permeable coating; multi-layer pharmaceutical forms, i.e. tablets in which the medicinal substances are applied as different layers by coating solutions, tablets in which the medicinal substances are distributed into different layers superposed by successive compressions, optionally separated by layers of excipients; microcapsules formed by elementary particles of small dimensions coated with a protecting film; tablets containing in their mass delayed-release microcapsules; pharmaceutical forms comprising spherical particles provided with dialysis membranes; capsules filled with subunits such as mini-tablets, pellets, and pillets containing the active constituents coated with layers of different ingredients of different thicknesses; osmosis configurations including ducted and bursting; diffusion; bioerosion; disintegration; and metabolism. An additional means of preparation is the boring of a prefabricated time-disintegration tablet or caplet to form a hollow space, filling with an active arousal compound, and re-sealing with a material which effectively restores the unity of the dosage form and re-enables its delayed-release function.

### Membrane Attributes for Effective and Reliable Operation

The length and fidelity of the delay interval is keyed to design of the inner subsystem. And, performance of the subsystem is most directly a product of qualities of the semipermeable membrane.

The foremost performance attribute for membranes is true semi-permeability, meaning that the layer should allow influx of water, or at least water vapor, but must strictly inhibit outward diffusion of core contents antecedent to rupture. As explained in the Background, all prior art osmotic bursting configurations exhibit leakage of active agent before rupture of the membrane. If leakage were to occur from a delayed wakeup formulation, the sleeping patient would begin to absorb the precociously delivered stimulant, disturbing rest, probably inducing terminal insomnia, and thus detracting from, or precluding, potential benefit.

The next most important membrane attribute is adequate strength, to withstand increasing internal pressure and prevent premature bursting, such strength being dependent upon film integrity.

An additional membrane quality is the ability to positively deliver the medicinal content to the g.i. tract all at once. Performance here is most specifically due to materials properties, especially film cohesion and elasticity.

Attributes of the water permeable yet insoluble membrane are chiefly an issue of chemical constitution of the particular film former. Materials for the membrane are, in general, the plastic film-formers, and may include polymeric cellulose derivatives such as ethers and esters. The performance requisites of the invention may often be attained by combining two or more different polymers. However, the scope of the formulation is not restricted either to any specific film-former or permutation of film-formers. Film integrity is a primary constitutional property of membrane quality, and is rated in terms of tensile strength as expressed by the Young's Modulus. Elongation is a correlative of film integrity, and change of shape is due to osmotic pressure. In best mode, the preparation must allow the membrane to stretch concurrently with hydrophilic expansion of the core contents. It is essential that the stretching proceed to the formulated extent without tearing, but that rupturing occur easily once that limit has been exceeded. Crosslinking, preferably extensive, is also a fundamental constitutional characteristic, and the degree directly relates to both membrane strength and resistance to erosion.

Yet another consideration in achieving membrane attributes is the solvent system. In creating membrane tissue, suspensions of polymer materials in aqueous liquids have been used with some success, but the integrity gained by solutions from volatile organic compounds (VOCs) has been observed to be exponentially superior, especially as regards prohibition of leakage.

### Core Attributes, for Effective and Reliable Operation

As previously set forth, the length of the delay interval is keyed to the design of the inner subsystem and properties of the semipermeable membrane. However, optimal membrane function is related to certain attributes of the cores, which in turn depend upon the technical method of fabrication, compositional materials, and resultant substrate physical characteristics.

A major decision must first be made as to whether the dosage form is to be configured as either a single subunit, e.g.—a conventional tablet, or as multiple subunits loaded into standard capsules or otherwise agglomerated. Once this decision has been made, the predominant variable affecting desirable physical core attributes is will be diameter. Complementary physical variables are surface area, density, and spheronicity. The corresponding essential attributes of suitably prepared cores are precisely appropriate diameter, minimal surface area relative to diameter, medium-to-high density, and superior rotundity. Second order core variables, namely temper and stability, concern structure. Materials properties such as binding strength and hardening capability most directly affect these, but may also influence the density variable.

Yet another physical variable is osmotic potential.

And, the final significant issue is bioavailability.

In addition to its obvious connection with volume, diameter has an inverse geometrically proportionate relationship with surface area. Furthermore, because the length of the delay interval is inversely proportional to the ratio of surface area-to-volume, diameter is the foremost quantity governing programmability of the delay.

In the current invention, possible embodiments include dosage forms with multiple small subunits. Preferably, such pillet or mini-tablet cores have relatively large diameters of from 1.5 mm to 3.2 mm. This diameter range enables minimization of surface area primarily by direct geometric relation with the volume which becomes large in proportion to the outer surface. Enlarged diameter further minimizes surface area by better control of spheronicity.

Remaining physical aspects of core surface area-to-volume ratio include those which involving the variable of surface area itself. Surface area is specifically affected by texture, and is closely correlated with rotundity. Thus, cores formed by compression tabletting should have smoothly rounded seams and edges. And, cores fashioned by other processes should display highly uniform spheronicity.

Medium-to-high density, with higher densities being optional, is generally the next most important core attribute, since mass-to-volume ratio is most likely to affect the outcome of the subsequent steps for making the dosage form. Materials content generally affects the degree of density, and properties to be most carefully considered are particle size and compressibility. The starting powders of active agent, binder, hardener, and any other additive should be milled so that the grains are smaller than 200 μm. The advantage is that interstitial spacing, i.e.—pathways by which moisture penetration can be more direct, is greatly diminished. As a result, the rate of influx through the semipermeable membrane is reduced, and the longer delay intervals requisite to the design are achievable.

Compressibility of core materials in tabletting processes is most easily enhanced, and thus density increased, by such reduction of particle size. In addition to increasing density, reduction of particle size may also have a bearing on osmotic function of the membrane. The force of compression also has a great influence on hardness and disintegration time. Having an inversely proportional effect on interstitial spacing, force accordingly has an inverse effect on rate of permeation. Hardness, on the other hand, has a directly proportional relationship to compression force, and is the chief factor concerning strength of the core interior.

Temper and stability are also factors of core strength, essentially involving binding and hardening excipients. Binders of choice is include polyvinylpyrrolidone (PVP) and its close variations. A preferred hardener for most of the technical methods of core construction is microcrystalline cellulose.

Equally important core materials properties are solubility and hydrophilicity, which affect rate of permeation. Whenever possible, the pharmaceutical active should reside in a form with decided orientation to become an osmotic solute, such as a medically beneficial salt. The total hydrophilic aspect of the core includes the characteristic solubility of the active agent in summation with solubilities of the excipients.

A highly preferable condition of cores which should exist upon rupture of the membrane is advanced bioavailability of the arousal medicament. As a result of circumspect design of the invention, this requisite is well met, because core contents become a partial solute while wicking proceeds up to the time of bursting. Thus, upon exposure to the lining of the digestive tract, the medicinal substance is already in a form conveniently suited for absorption.

### Means for Predetermination of Length of Delay Interval

The specific length of the overall lag time can be programmed best by adjusting the time segment between the chronological point at which the subsystem commences the intermediate phase and the time when its subunits undergo bursting. The first definitive factor is the rate of permeation of water through the semi-permeable membrane. In turn, a primary modulator of this rate is the permeability of the skin-like membrane. Permeability can be delimited most directly by choice of polymer type from which the membrane is constructed. Substitutions here enable large variations in the delay before release. Among the exemplary membrane materials most preferred for their ease of use and adaptability are cellulose ethers, organic cellulose esters, inorganic cellulose esters, and the acrylic resins. A standard rating method for polymer films is the water vapor transmission rate (WVTR), tables for which are found in “The Guide to Plastics,” a supplement of Modern Plastics magazine. Permeation is occasionally further modified to a lower rate by adding permeation retarding materials to the coating mix. These substances may include fatty acids, waxes, and the salts of the fatty acids such as magnesium stearate and calcium stearate, as well as fumed silica (Aerosil® R972 by Degussa).

Finer-scale alterations to extent of the delay interval are most easily achieved by varying the thickness of the membrane. If the dosage form is prepared so that the membrane is thicker or thinner, there will be a corresponding increase or decrease of the pathway distance through which water must travel. Accordingly, the permeability rate will change by inverse proportion. Small variations to the delay time are also workable by changing the radius of the core-shell subunit. If the core radius is modified, there will be a direct and geometrically great change in the entire surface area of the enveloping semi-permeable membrane. This new total area will augment or reduce the sum penetrability, resulting in an increase or decrease of the rate of permeation of water into the dosage form. The relationship which is affected is the surface area-to-volume ratio. Modification of radius is generally accomplished by varying the amount of bulk material, such as filler, which is combined into the core composition. Note: Diversifying membrane materials, altering thickness, and change of radius all have secondary effects on the capacity volume of water required to burst the membrane.

One further means for altering the rate of permeation, and thus the length of the delay interval, is to supplement the existing osmotic pressure of the pharmaceutically active agent by combining one or more excipients known as osmotic attractants into the core during fabrication. By addition of such substances, augmentation is imparted to the osmotic potential of the inner side of the gradient, thus accelerating the induction of water. From this last provision, preparations can be arranged for availing pharmaceutical actives for which the dosage form would otherwise be impracticably limited by weak osmotic pressure. Typical groups of osmotically attractant excipients are binders, swelling agents, and disintegrants. Compositions are not, however, restricted to these few materials cited. As a tactical variation, excipients may be incorporated which, apart from being hydrophilic, create relatively dramatic increases of pressure by evolution of carbon dioxide gas.

In addition to rate of permeation, the other definitive factor for programming duration of the delay interval, is the capacity volume of water which is required to burst the subunit membrane. Besides surface area to volume ratio as discussed supra, modification of core-shell radius will also change the collective dimensioning of the entire dosage subunit, resulting in a great change of volume, which in turn alters the amount of water which must be absorbed in order to stress the membrane beyond its bursting point. As all subunit shapes deemed acceptable in this invention have a cylindrical or ellipsoidal axis, variance of radius will very predictably increase or decrease the total volume of water required to distend the membrane beyond its limit. The corollary will be a corresponding change in the amount of time which passes anterior to bursting. Further aspects of capacity water volume are the toughness of the membrane tissue and its tolerance for distention. As with permeation rate, these qualities depend on the particular materials selected for composition of the membrane, and also its thickness. Toughness increase or decrease, imbued into the membrane tissue by changing the film former, will be reflected as a different tensile strength rating. Furthermore, selection of a certain polymer for its higher or lower elongation value will correspondingly set the duration of the delay interval, for longer or for shorter. Math formulas for predicting delayed-release behavior are discussed in U.S. Pat. No. 3,247,066.

Generally, the range for the overall lag, from the time of ingestion to the initiation of stimulus action, is approximately 5 to 9 hours and preferably about 5 to 7.5 hours. Concordantly, the length of the time segment between the prompt release of sleep-compatible substance and delivery of arousal agent, in basic embodiments, falls within about 4.5 to 8.5 hours. Optimally, the programmed delay falls between about 4 to 7 hours. One version for these ranges is contemplated wherein the planned interval of sleep is a nap. In this situation, the overall lag may run from 2 to 5.5 hours, based upon a programmed duration for the delay falling between about 1.5 and 4.5 hours. It should be noted, however, that these time lengths may vary by design to accommodate the individual needs of diverse classes of patients.

### Wakeup Agent and its Final Delivery

At the end of the delay phase, the membrane bursts, and components of the core are affirmatively and completely discharged to the gastrointestinal tract. The conclusional pharmaceutical action begins following absorption of the arousal agent, and delineates the end of the sleep interval. The response sequence, via neural stimulation, proceeds dramatically with a rise in motor potential, accelerated heart rate, deeper inspiration, and stirring of the patient, closely followed by transition to full consciousness, and expected exit from bed.

The wakeup component will generally include at least one pharmaceutically active energizer, invigorant, or nervous system stimulant, such as amphetamine, methylphenidate, venlafaxine HCl, nefazodone, sodium oxybate, adrafinil, modafinil, phentermine, and pemoline; xanthines including theophylline, theobromine, and caffeine; serotonin reuptake inhibitors and agents having similar mode of action; substances pending release due to further development, ongoing clinical trials, FDA review, or other protraction; drugs temporarily withdrawn from the market or otherwise unavailable; as well as the close relatives and derivatives of any members from this group; and any combination of these recited agents is also subsumed. Those agents used for treatment of Attention Deficit Disorder and Attention Deficit Hyperactive Disorder, having pharmaceutically stimulating properties, may also be adapted. Possible arousal agents for the formulation are by no means, however, confined to those mentioned here.

All wakeup agents proposed for formulation have posologies which are associated with negligible toxicity, thus an all-at-once, or “pulse” release is both appropriate and prudent. Upon absorption, chemical stimulants induce ventilatory, cardio-vascular, and other energizing phenomena which are well known as being reversive to the normal course of sleep. Arousal agents trigger such effects either by direct action on the nervous system else by activation, disinhibition, or potentiation, of endogenous invigorating hormones.

The new invention is especially therapeutic for disorders wherein symptoms include detrimental patterns such as sequences or continuing rounds of sleep inadequacy accompanied by difficulty in awakening. The sleep regulating system can breach such negative circles, by improving punctuality, and reinforcing employment surety. Certain SDs, including Hypersomnia and Sleep Paralysis, benefit in that not only is difficulty in making transition from sleep to wakefulness addressed by the conclusional release of arousal agent, but the overall sleep cycle is harmonized by the integral formulation. This comprehensive improvement is achieved in some measure by the first release, the calmative or other agent of which helps in overcoming any incidental initial insomnia. Such insomnia is frequently a contributing source, and occasionally a principal cause, of such SDs which may not overtly reveal insomnia as a symptom.

Lingering effects of stimulant usage can cause problems if the agent is ingested by ordinary means, such as drinking coffee, late in the day or in the evening. In contrast, as provided by the subject innovative dosage form, residual arousal agent in the blood plasma of an individual, in prime hours following wakeup, can be beneficial. This remaining agent can aid alertness during early activities. As a leading example, the protracted vigilance helps while an individual operates a motor vehicle, thereby reducing probability that the driver will become involved in a traffic accident. Residual stimulant can also energize productivity in early work pursuits for a limited time.

The mean half-life of caffeine in plasma of healthy individuals is about 5 hours, according to Brachtel and Richter, as well as Busto et al. From a gathering of other sources, the opinion for elimination half-life falls between 1.5 to 12 hours, averaging about 3 to 5 hours. Thus, a considerable blood level of caffeine will persist for a minimum of nearly two hours, and more often for over four hours. Likewise, methylphenidate is a short acting stimulant that has a half-life ranging from 2 to 3 hours and averaging 2.5 hours (Kimko, et al).

Amphetamine has a half-life which varies from about 4 to 15 hours, averaging 10 hours (Shire), presenting the benefit of prolonged support to productivity into mid-day work activities, but perhaps longer than optimal for complete avoidance of late day stimulation. Similarly, the pharmacokinetics of modafinil are characterized by a lengthy half-life of approximately 15 hours (Wong, et al). But, for any of these stimulants, a driver who commences transit to his destination shortly after being stimulated to wakefulness will quite certainly be alert during a journey lasting up to a few hours.

In the current conception, the preferred release configuration for any wakeup agent is, pursuant to the delay phase, prompt and complete delivery. Sustained release is generally unnecessary, attributable to the benefits of residual action.

The invention serves furthermore as therapy for insomnias, and other SDs which include insomnia as a manifest symptom, in that not only is initial insomnia addressed by release of calmative or other agent which is compatible with drowsiness and slumber, but the overall sleep cycle is symphonized by the integral formulation. For these disorders, the comprehensive improvement is achieved collaboratively by the conclusional release, wherein pursuant to assisting wakeup, residual arousal agent promotes punctuality and boosts vigor in early waking activities. Prompt awakening and the post-wakeup benefits essentially frame a strong start, universally acknowledged as a critical determinant of good effectiveness early in the day. Since afflicted individuals are then less anxious about accomplishing a satisfactory amount of productivity for the day, the treatment reduces compulsions to ingest stimulants after noon and immerse in work or other agitating engagements proximate to bedtime. Thereby, potential sources of insomnia are trimmed, diminishing incidence of sleep inadequacy.

In the case of either insomnia-symptomatic SDs or those which may not overtly demonstrate insomnia as a symptom, such as Hypersomnia and Sleep Paralysis, variant formulations may provide an interim release of calmative or other agent to address middle insomnia rather than, or as well as, initial insomnia.

Up to the present, no direct pharmaceutical treatment has been available for stress. Instead, drugs have been prescribed for treatment of anxiety, which typically includes generalized anxiety, acute anxiety, clinical anxiety, and chronic anxiety. This practice is somewhat flawed, as anxiety may be only one component of stress. By giving rise to insomnia, sleep deprivation, and consequent difficulty with wakeup, stress can often cause tardiness and absenteeism. Not infrequently, a continuous state of overstress and precarious employment develops.

The loop is disruptable by improving startup and thus reducing tardiness. Since, by action of residual arousal agent, the new sleep regulating system improves initial organization pursuant to wakeup, better punctuality and reduced absenteeism are experienced. As in results for DSPS and CFS, the performance gain improves job or career security, thereby allaying stress and dispersing the negative circle. Rather than just attempting to treat symptoms, the invention deals with the very source of stress, namely, vicissitude in endeavors to attain employment success and stability. This novel method of treating stress may alternately be conducted in conjunction with other therapies, including anxiolytic drugs as well as non-pharmaceutical stress management strategies. Such non-drug approaches may consist of psychotherapy, physical exercise, and relaxation techniques, either singly or in any combination.

Depression therapy, as heretofore established, has consisted primarily of administration of antidepressants, thus certain aspects of mental well-being have not been addressed. Elements such as productivity and feelings of efficacy have been practically ignored. As in treatment of stress, depression, and CFS, the new pharmaceutical supports energetic accomplishment in early hours following wakeup, and thus promotes a hale sense of personal adequacy and competence. Other elements may also be improved. By fortifying these factors, psychological health is balanced, and thus the actual causes of the depression may abate. In addressing these qualities of positive self-concept, the invention is highly innovative. The formulation is valuably pliable as either adjunct or primary therapy for depression. Moreover, as stress and insomnia have a definite relationship, and in recognition that insomnia is commonly an indicator of depression, the medication is particularly indicated when stress is accompanied by depression or vice versa.

Chronic Fatigue Syndrome, thus far, has no proven effective treatment. At the very least, pursuant to a prompt wakeup, the invention can relieve CFS symptoms in early waking activities. As in the above-described treatments of other medical conditions, residual arousal agent improves early vitality, which in this case countervails feelings of overwhelming weariness. By improving alertness, the medicine also affords safety to afflicted individuals by reducing probability of drowsing or falling asleep while driving or otherwise traveling to work. These benefits are conducive to punctual arrival, and can significantly reduce the high probability (50%) of job loss faced by CFS sufferers due to their incapacitating symptoms. The sleep regulating system can serve as an adjuvant to other CFS therapies as may become available. Alternatively, the formulation can be administered independently.

### Technical Methods for Fabrication

### Core Creation:

The first step for preparing the new medication, may be accomplished by several methods, including:

Colloid Gelatination—In this method, the starting substance is heated until liquid, then cooled suddenly, resulting in formation of spheres of almost perfect uniformity. A typical routine is disclosed in U.S. Pat. No. 3,092,553. Another process for forming very small cores, e.g. microspheres, by colloid spheronization is explained in U.S. Pat. No. 5,718,921.

Compression Tabletting—For standard-sized dosage forms with approximate subunit volumes of 0.001 cc to 1.0 cc, one basic technique for fabrication is conventional direct compression tabletting as customary in the pharmaceutical industry. The dimensional range for such cores includes mini-tablets (typically later filled into capsules) through full-sized tablets of 5 mm or more. Preferred tooling shapes include spherical punch with socket type die, and modified ball.

Extrusion-Spheronization—This technique is relatively common in the art, but is modified as applied to the invention. Spheroid cores for testing may be prepared in equipment adapted to R & D scale. As a representation, wet granulation may be conducted in a Kitchen-Aid K45SS Stand Mixer, and then extruded with the grinder attachment through a specially modified “pasta” die with enlarged (e.g.—3 mm) bores to give an extrudate which is subsequently spheronized by a merumerizer machine (Fuji Paudal) then dehumidified in a fluid bed drier.

Buildup onto Inert Cores—Prefabricated sugar spheres, known in the industry as “Non-pareils,” are supplied in different grades, the preferred size for the present design being somewhat large. Other inert cores, comprised of various excipients, may likewise be used as starter seeds. The first coating application is from a solution which contains the active agent, resulting in medication-layered cores which are subsequently processed for delayed release.

Rotor Granulation—Fabrication begins in the processing chamber of a rotor granulator apparatus with a dry powder, the mix of which includes the active agent. The particles are blown airborne while a liquid binder is sprayed into the chamber. Agglomeration builds the powder into pellets with fairly well-rounded edges, homogenous size, and good spherical uniformity. Thus the wakeup medication is integral with the core body, rather than layered on. This type of equipment benefits from a very effective interplay of buoyancy, gravitation, and centrifugal force, thus rotating the cores in a spiral, torus-like motion.

Direct Pelletization—This method is very similar to rotor granulation. In fact, the technique is conducted within a rotor granulator-processor machine. However, the final objective differs. The primary goal in direct pelletization is the production of dense, exceptionally spherical, larger pillets with minimal interstitial void space and increased strength. Basically, the routine begins with an airborne dry powder, including active agent, in the processing chamber. Binder is tangentially sparged into the chamber with the nozzle positioned in the densest portion of particle movement. Spheronization is accomplished to a large extent by varying certain apparatus assemblies and parameters which develop a high humidity within the chamber. Also, disk surfaces are selected which effect a higher shear than those typically used for rotor granulation. The resulting cores have little or no proclivity to capillary wicking, and thus do not detrimentally accelerate permeation through the membrane of the finished dosage form.

Specific procedures for incorporating wakeup agents and excipients into cores are disclosed in the Examples.

### Membrane Fabrication

In the general application of polymer lacquers for creation of membranes, a spray-coating solution is applied to the cores by a fluidized bed apparatus. Due to its operational requisites, the invention is oriented to high performance plastic film-formers, solvents for superior spray-coating solutions of which frequently consist of VOCs.

Preferred film-forming polymers include organic cellulose esters such as, but not restricted to cellulose acetate (CA), cellulose acetate formate (CAF), cellulose acetate propionate (CAP), cellulose acetate butyrate (CAB), cellulose acetate pentanoate, cellulose acetate benzoate, and cellulose triacetate; inorganic cellulose esters such as, but not limited to, cellulose nitrate (CN, a/k/a nitrocellulose), cellulose sulfate, cellulose phosphate, cellulose phosphite, cellulose halogenides, cellulose borate, cellulose titanate, and cellulose xanthate; cellulose ethers such as, but not confined to, ethylcellulose and ethyl hydroxyethyl cellulose; polyvinyl alcohols; polyurethanes; and nylons; as well as vinyl esters such as ethylene vinyl acetate (EVA), polyvinylacetate (PVA), and polyvinyl butyrate. Additional film-forming polymers for membranes include methacrylic acid/methyl methacrylate copolymer (MA/MMA). Non-limiting combinations of compatible polymers may comprise EC with CAB; CAP with CAB; polycaprolactones with either PVAc or CN; EVA with CN, and EC with CN.

If ethylcellulose (EC) is chosen for the membrane material, viscosity classification and ethoxy content both will have a significant bearing on finished coating quality. Dow's Ethocel Medium-50, with viscosity ranging from 45 to 55 cP and ethoxy from 45.0 to 47.0%, conforms to criteria for integrity and is also approved for pharmaceutical use in accordance with the N.F. and U.S.P. Superior results may be obtained by using VOCs in certain combinations, such as, but not confined to, co-solvent blends of lower alcohols with aromatic hydrocarbons.

Preferred plasticizers useful for incorporation into the membrane include, but are not restricted to: diethylene glycol dibenzoate; diisobutyl adipate; sebacates such as diethyl sebacate, dibutyl sebacate, and dioctyl sebacate, phthalate esters such as dimethyl phthalate (DMP), diethyl phthalate (DEP), dibutyl phthalate (DBP), dioctyl phthalate (DOP), diisooctyl phthalate, and di-2-ethylhexyl phthalate; as well as the citrate esters such as acetyltriethyl citrate (ATEC), tributyl citrate (TBC), and acetyltributyl citrate (ATBC). Other valuable film-formers, combinations, and plasticizers are shown in the Examples.

Generally, spray processing within a Wurster-style fluid bed apparatus, wherein abrasive forces are minimal, is best for application of the thin membrane coatings. An excellent choice is the model GPCG-3, for R&D, with 6″ Wurster column and RI-300 rotor insert, by Glatt GmbH, Germany. Machinery parameters for membrane preparation by fluid bed include humidity, drying rate, air velocity, application rate, temperatures, and nozzle atomization pressure. If core creation is by direct pelletization in a roto-granulator apparatus, once the desired diameter is reached, processing may be continued after merely halting rotation of the disk. Meanwhile, fluidization is maintained, and spray application of the membrane coating solution can be initiated.

The next stage of the fabrication series is integration of the outer component group. Basically, this is comprised of a sleep-compatible layer, fashioned similarly to most simple coatings, but which may carry some substance of a pharmaceutically calming or tonic nature. The layer is generally configured so as to disintegrate quickly by simple dissolution, to release drug immediately and enable rapid onset of drowsiness. Any method for applying the calmative layer to the subunits should be a minimally abrasive process.

Spray solutions for the calmative coating are conveniently formulated by mixing the active agent with erodible, hydrophilic binder excipients, such as hydroxypropylmethylcellulose (HPMC), polyvinylpyrrolidone (PVP), and xanthan gum, and are ordinarily applied directly to the previously prepared membrane layer. The outer component group may include any combination of outermost protective or beauty coats covering the sleep-compatible layer. Optional arrangements for incorporation of the calmative component include press-coating onto the coated cores, as well as powder fill into conventional capsules wherein multiple coated cores are loaded.

The specific sleep-compatible agent may be selected from tonics, calmatives, hypnotics, muscle relaxants, sedatives, anxiolytic agents, anti-insomnia agents, tranquilizers, hormones, endorphins, or similar medications. Also included are herbal extracts and substances traditionally reputed to have soporific effects, such as but not confined to valerian and hops. Non-limiting representatives of preferred agents are benzodiazepines such as temazepam, lorazepam, triazolam, alprazolam, cloxazolam, estazolam, etizolam, haloxazolam, bromazepam, clonazepam, clotiazepam, diazepam, fludiazepam, flunitrazepam, flurazepam HCl, halazepam, medazepam, nimetazepam, nitrazepam, oxazepam, quazepam, and olanzapine. Other possibilities include non-benzodiazepines such as risperidone, zaleplon, zolpidem tartrate, L-tryptophan, 5-hydroxy-L-tryptophan, melatonin, diphenhydramine, and doxylamine succinate. Furthermore, it is anticipated that new pharmaceutical agents which are still in development or testing, such as but not restricted to, eszopiclone ((S)-Zopiclone) by Sepracor, indiplon (NBI-34060) by Neurocrine Biosciences, Inc., NGD 91-2 and NGD 96-3 by Neurogen Corp., as well as NS2710 by Neurosearch A/S, will be desirable for various future formulas.

Moreover, “sleep promoting factor,” for which research was originally pioneered independently by both Kuniomi Ishimori in Japan and Henri Pieron in France and set forth in their publications of 1909 and 1913 respectively, is encompassed within the possible active substances.

### ALTERNATIVE EMBODIMENTS

Other possibilities for organizational structure and operation of the dosage form may consist of arrangements wherein delay is effected by a combination of means or subsystems. One alternate configuration is comprised of an agglomeration of multiple subunits such as pellets and granules surrounded by a main semi-permeable membrane. Optionally, the small subunits themselves also may each be coated by individual membranes.

Other alternative embodiments involve versions wherein it is advantageous during the delay phase to release one or more complementary substances, such as, but not restricted to, medications for treatment of sleep apnea, synergizing agents, additional portions of the initially released agent or other sleep-therapeutic substance, and vitamin complexes. These complements are distinctly different in action to the final agent. Means for interim release of such substances may include configurations of multiple membranes organized to burst in series, an innermost membrane being responsible for containment and ultimate release of the wakeup agent.

### Ramifications

Eventual introduction of new, refined stimulants into the pharmaceutical field is anticipated, and these agents will be considered for incorporation into the invention.

One category of arousal agent is utilizable upon special preparation. The natural stimulant adrenaline presents a great problem in adaptation to pharmaceutical applications. Adrenaline is rapidly degraded by blood enzymes, and any therapeutic level which is attained will be diminished almost completely within a few minutes. Thus, for use as a wakeup agent, an improvement must be organized to prolong the resident effective blood level at least far enough to allow the patient to react and, preferably, exit the bed. One useful provision would carry an auxiliary short-sustain subsystem within the primary subsystem of the oral dosage form. This configuration could be comprised of a plurality of mini-subunits such as microcapsules or nanoparticles which, pursuant to absorption, would progressively release the adrenaline over a therapeutically sufficient time period. The range of agents in this special category includes the natural stimulant adrenaline, as well as its derivatives, relatives, and analogues. Furthermore, as mentioned above, sleep-suppressive stimulation can be induced by activation, disinhibition, or potentiation of endogenous energizers. An encompassed arrangement, therefore, would be the mobilization of intrinsic adrenaline by release of an endocrine or chemical agent which acts on the adrenal medulla. Other arrangements could operate by interfering with restrictions on adrenaline effects, through agents such as cyclic adenosine monophosphate (CAMP). Whatever the particular design, the span of time for this tactical short sustain would simply be that which is sufficient to achieve the necessary response from the patient, e.g.—about 10 to 20 minutes, with further sustain optional in formulations where targeted disorders call for improvement of alertness and vigor in early activities.

The scope of the invention is extendable to therapies for conditions seemingly unrelated to sleep disorders. In a nap-styled formulation, short-acting stimulants such as adrenaline would be especially valuable for treatment of medical conditions such as night incontinence, since the exceptionally brief half-life would allow the sufferer to fall asleep again after taking measures to prevent an incontinent episode. Similar configurations may be therapeutic for sexual disorders, which are mitigated by relaxation. In this application, since the treated individual would be asleep, up to the time of wakeup, provision for relaxation would be as complete as imaginable. For such disorders, complex formulas are encompassed in which another substance is included with the release of stimulant but having a separate therapeutic action pursuant to wakeup. Options for such a substance include, but are not limited to, treatments for impotence and poor libido.

Sophisticated variations of the central plan may incorporate yet another different agent which would be released along with the stimulant, and whose purpose would be to effectively abbreviate the half-life of any residual from the previously released calmative. One mode of action for such an agent could entail mop-up of remaining blood levels of the drug. Another means of operation is interference with the action of whatever level of calmative agent may remain in the plasma. An applicable anti-hangover agent would be flumazenil, upon development in an orally administerable form, for blocking unwanted lingering effects as may be present from benzodiazepines. Other modes of action are also possible.

Since the low side of Bipolar Disorder (formerly known as Manic-Depressive Syndrome) resembles depression, the pharmaceutical may eventually reach to treatment of this problem. Similarly, because Attention Deficit Disorder (ADD) and Attention Deficit Hyperactivity Disorder (ADHD) respond so well to stimulants, the pharmaceutical may ultimately be adapted for these problems as well.

Perhaps the most remarkable enhancement contemplated for the invention is user adjustability. By this provision, the dosage form can be “set” in a manner somewhat like an alarm clock. With integration of such an operational control, variable duration of the delay interval will be enabled. As would be expected with a conventional manufacturing sequence, the preparation may be dressed with any range and combination of outer protective topcoats or “beauty” coatings. However, a fortunate opportunity is presented at this point for a provision which would enable custom setting, just before administration, of the length of the delay interval. As a simple means for imparting leeway in the starting point for the delay phase, the protective coat can itself be composed of materials and structure which allow coupling with the delay function. One elementary arrangement would be embodied with a removable exterior covering comprised of a capsule cap and body with geometry essentially identical to that of a common gelatin capsule. This outer cover would have a character such that it would erode at a moderate pace over, for instance, a one hour period. Thus, there would be a preliminary retard stage in sequence with the delay interval as already provided by the bursting membrane, the total time constituting the overall lag. With this accessory, selective removal and discard of the covering halves could be optioned by the patient. In unremoved mode, the outer covering would disintegrate slowly, and overall lag time would be the unattenuated nominal 7.5 hours. Or, upon discard of the covers and ingestion by the patient, there would follow a delay phase which would be shortened by one hour. Thus, the overall lag would become 6.5 hours.

Alternative embodiments for the removable cover could include peelable layerings. Moreover, combinations offering second and possibly further stages of postponement are possible for tailored re-programming at the discretion of the patient. It should be noted, however, that user-adjustability as to duration of delay would not be bound to removable or peelable covers, or any other particular design. The configuration may encompass any means by which a patient would be able to, preceding ingestion of the dosage form, accomplish adjustment for a specific delay. Future sophisticated controls may include lengthening of the delay interval as well as shortening, and optional adjustment of the release of calmative.

### EXAMPLES

### Example 1

### Caffeine Cores Coated with Ethyl Cellulose Membrane, and Melatonin Calmative

### Preparation of Caffeine-Containing Cores by Direct Pelletization

A suspension was prepared according to the following table:

| | | Subunit | Dosage | % total | Batch |
|---|---|---|---|---|---|
| Ingredient | Grade/type | Wt., mg | Wt., mg | dose wt. | wt, g |
| Caffeine, USP | anhydrous, 99% | 4.00 | 100.0 | 51.3 | 150.0 |
| Microcrystalline | Avicel PH105 | 2.00 | 50.0 | 25.6 | 75.0 |
| cellulose | | | | | |
| Calcium | Pharma-Carb LL | 1.28 | 32.5 | 16.7 | 48.0 |
| carbonate | | | | | |
| Copolyvidonum | Plasdone S-630 | 0.56 | 14.0 | 6.4 | 21.0 |
| | | 7.84 | 195.0 | 100.0 | 294.0 |

### Mixing Procedure:

Microcrystalline cellulose (FMC Corp.), 75.0 g, and calcium carbonate (DMV Pharma, average particle≦20 μm), 48.0 g, were combined in the chamber of an Atritor Microniser Model 2 spiral jet mill, then simultaneously comminuted and mixed for 20 minutes.

The binder, N-vinyl-2-pyrrolidone/vinyl acetate copolymer 60:40 (Plasdone S-630, ISP), was dissolved in warm water at a temperature of about 50° C. The remaining amount of water was then added under stirring, for a total water contribution of 2.7 liters. The wakeup agent, 1,3,7-trimethylxanthine (caffeine, Pfizer Food Science Group), the microcrystalline cellulose, and the calcium carbonate were then added to the mixture under stirring. The resulting mixture was passed through a #80 sieve immediately before use. The resulting suspension was manually stirred at regular intervals to ensure a homogen feed.

The suspension was sprayed into the chamber of the RI-300 rotor granulator module of a Glatt GPCG-3, the main system having mechanical modifications to increase air velocity and thus enable fluidization of larger cores. A variable rate peristaltic pump provided spray impetus. The apparatus was also furnished with a 90 psi, 120 gallon compressed air supply to enable long process times. Nozzle position was tangential. The selected rotor disk had a medium-fine grade surface. The following parameters were employed:

| | Time, minutes | | | | | | | |
|---|---|---|---|---|---|---|---|---|
| | 0 | 30 | 60 | 90 | 120 | 150 | 180 | 210 |
| Inlet | 57 | 60 | 67 | 67 | 67 | 67 | 67 | 43 |
| temperature, | | | | | | | | |
| ° C. | | | | | | | | |
| Product | 52 | 55 | 62 | 62 | 62 | 62 | 62 | 40 |
| temperature, | | | | | | | | |
| ° C. | | | | | | | | |
| Outlet | 40 | 43 | 46 | 46 | 46 | 46 | 46 | 29 |
| temperature, | | | | | | | | |
| ° C. | | | | | | | | |
| Application | 10 | 15 | 15 | 15 | 15 | 15 | 15 | — |
| rate, g/min | | | | | | | | |
| Atomization | 1.8 | 2.8 | 2.8 | 2.8 | 2.8 | 2.8 | 2.8 | — |
| pressure, bar | | | | | | | | |
| Rotor speed, | 150 | 200 | 300 | 500 | 800 | 800 | 800 | off |
| rpm | | | | | | | | |
| Air flow rate, | 80 | 90 | 110 | 135 | 165 | 175 | 175 | 160 |
| m³/hr | | | | | | | | |

Agglomeration began immediately, and as the small granules hit the rotating disk, they were compacted and rounded. Rather than a deposition and build-out of prefabricated seeds as in Example 3, the agglomerating material in this process becomes its own substrate, and is further spheronized as the granules slowly grow into dense pellets.

Humidity was elevated by steam injection. Because much of the heat is contributed by this provision, the inlet air temperature could be set to a reduced level as compared with processes relying entirely on electric heating of the air. The steam was shut off after 210 minutes.

Once all of the suspension was consumed, the rotor was likewise switched off but fluidization was continued for 20 additional minutes at a product temperature of 35-40° C. to dry the cores. The batch was further dried for 6 hours in a conventional oven set at 40° C. to remove as much of the remaining moisture as possible. The batch was next passed through a special #8 sieve, made with 0.635 mm (0.0250″) wire, and having 2.54 mm holes (Mahaveera Industries, Navi Mumbai, India), to remove oversized spheres. Then the product was screened with a second special #8 sieve made with 0.711 mm (0.0280″) wire, and having 2.46 mm holes (Newark Wire Cloth), to remove undersized product and fragments. Approximately 10,000 subunits, weighing 78 grams, were culled. The net yield was about 20,000 drug-loaded cores, with core diameter very closely approximating 2.5 mm and weighing 7.84 mg each.

The coating apparatus, including the spray nozzle, tanks, pumps, and lines were cleaned thoroughly in advance of the next step.

### Formation of Ethyl Cellulose Membrane by Application in Wurster Column

A coating solution was prepared according to the table below:

| | | Subunit | Dosage | | |
|---|---|---|---|---|---|
| | | Wt., | Wt., | Layer | Batch |
| Ingredient: | Grade/type: | mg: | mg: | % wt.: | wt, g: |
| Ethyl cellulose | Ethocel Med 50 | 0.24 | 4.8 | 80.0 | 4.8 |
| Dibutyl phthalate | DBP, 99.2% | 0.03 | 0.6 | 10.0 | 0.6 |
| Tributyl citrate | Citroflex C-4, | 0.03 | 0.6 | 10.0 | 0.6 |
| | 99.6% | | | | |
| | Subtotals | 0.30 | 6.0 | 100.0 | 6.0 |
| Caffeine cores | | 7.80 | 156.0 | | 156.0 |
| | Totals | 8.10 | 162.0 | | 162.0 |

Approximately 19,900 of the previously prepared caffeine-loaded pillets were loaded into the High Speed (HS) Wurster column of the Glatt GPCG-3. The nozzle was set in the bottom position. The spray coating solution was prepared from 4.8 grams ethyl cellulose (DOW, Medium ethoxyl content, 50 cPs viscosity, Premium grade), 600 mg dibutyl phthalate (Eastman), and 600 mg tributyl citrate (Morflex, Inc.) dissolved in a 1.5 liter solvent system of 25% ethanol (Absolute, USP Grade, 99.96%, Pharmco): 40% toluene (99.5% pure, J. T. Baker): 35% meta-xylene (m-Xylene, 99+%, anhydrous, Sigma-Aldrich) to form a semipermeable coating.

The following equipment parameters were used:

| | Time, minutes | | | | | | | |
|---|---|---|---|---|---|---|---|---|
| | 0 | 20 | 40 | 60 | 80 | 100 | 120 | 140 |
| Inlet | 45 | 45 | 47 | 48 | 50 | 50 | 50 | 45 |
| temperature, | | | | | | | | |
| ° C. | | | | | | | | |
| Product | 30 | 30 | 31 | 32 | 33 | 34 | 34 | 30 |
| temperature, | | | | | | | | |
| ° C. | | | | | | | | |
| Outlet | 25 | 25 | 26 | 27 | 28 | 28 | 28 | 25 |
| temperature, | | | | | | | | |
| ° C. | | | | | | | | |
| Application | 8 | 8 | 10 | 12 | 15 | 15 | 15 | — |
| rate, ml/min | | | | | | | | |
| Atomization | 1.8 | 1.8 | 2.0 | 2.5 | 2.8 | 2.8 | 2.8 | — |
| pressure, bar | | | | | | | | |
| Air flow rate, | 175 | 175 | 175 | 175 | 175 | 175 | 160 | 150 |
| m³/hr | | | | | | | | |

The spray step was started at a reduced rate, then accelerated more aggressively. All spray solution was consumed after 120 minutes. Fluidization was continued with reduced intensity for 10 additional minutes, at a product temperature of 25-30° C., to dry the cores. The gross yield of 20,000 coated subunits was first passed through yet another special sieve, #7 mesh, woven of 0.889 mm wire (0.0350″) and having 2.7 mm holes (Mahaveera), to remove agglomerates and oversizes. Then the batch was passed through the previously employed second special #8 sieve to remove particulates. About 5,200 subunits, massing 49 g, were culled as flawed or imperfect. The net was about 14,800 subunits weighing 8.1 mg each with average core diameter of 2.5 mm. The thickness of the membranes was approximately 18 microns.

### Application of Prompt-Release Melatonin Calmative Layer

Proportions for dry matter of the calmative-containing coat were as per the following table:

| | | Subunit | | | |
|---|---|---|---|---|---|
| | | Wt., | Dosage | Layer | Batch |
| Ingredient: | Grade/type: | mg: | Wt., mg: | Wt. %: | wt, g: |
| Melatonin | 98.5% pure | 0.16 | 3.2 | 64.0 | 1.6 |
| Povidone USP | Plasdone ® C-15 | 0.02 | 0.4 | 8.0 | 0.2 |
| Spray Talc | 99% < 25μ | 0.07 | 1.4 | 28.0 | 0.7 |
| | | 0.25 | 5.0 | 100.0 | 2.5 |

| | | | |
|---|---|---|---|
| Previously coated cores | 8.10 | 162.0 | 60.0 |
| Totals | 8.35 | 167.0 | 62.5 |

Purified water USP, 480 ml, and ethanol (Pharmco), 240 ml were admixed. This co-solvent system was warmed, and the batch of melatonin (Seltzer Chemicals) was added and mixed for approximately 20 minutes. While maintaining the solution at 44° C. and with continuous stirring, the polyvinylpyrrolidone (Povidone USP, from ISP) was added, and mixing continued for 10 minutes. The final constituent, spray talc, (Snowtalc Ultra, from Micronized Group), was added and mixing resumed for 30 more minutes.

The Glatt GPCG 3 apparatus was unchanged from its component configuration in the previous step. The HS Wurster column was loaded with 62.5 g of previously prepared subunits, and preheated until the bed reached approximately 38° C. Machine parameters were as follows:

| | Time, minutes | | | | | |
|---|---|---|---|---|---|---|
| | 0 | 15 | 30 | 45 | 60 | 75 |
| Inlet temperature, ° C. | 52 | 52 | 55 | 55 | 57 | 50 |
| Product temperature, ° C. | 36 | 36 | 37 | 37 | 38 | 45 |
| Outlet temperature, ° C. | 28 | 28 | 29 | 29 | 30 | 30 |
| Application rate, ml/min | 10 | 12 | 13 | 15 | 15 | — |
| Atomization pressure, bar | 1.6 | 2.0 | 2.5 | 3.0 | 3.0 | — |
| Air flow rate, m³/hr | 160 | 175 | 180 | 180 | 180 | 165 |

The entire calmative-binder solution was sparged onto the subunit batch. After fluidized drying for an additional 15 minutes, the batch was removed from the granulator and any agglomerates and fine powder were separated. Finally, the finished pillets were loaded into conventional #0 standard gelatin capsules, 25 subunits per each.

An additional lot was run with process identical to that described supra, except having one half the batch quantities of the above, and with substitution of FD & C Blue #2 pigment instead of the melatonin.

### Testing

Samples from the FD & C Blue #2 batch were tested in vitro for performance of the prompt-release layer in a type 2 dissolution apparatus (paddle) according to U.S. Pharmacopoeia XXIII at 37° C. in simulated intestinal fluid at 100 rpm. Release of the FD & C Blue #2 dye was detected at 12 minutes by direct visual observation, and was obviously complete within 20 minutes.

An automated spectrophotometry device (Beckman-Coulter) was then set up to monitor the delay progress and eventual release of the wakeup agent in the same dissolution testing apparatus. Caffeine was determined at 273 nm. Interpretation of the data showed the profile as per the table below:

| | Minutes | | | | | | | | | | |
|---|---|---|---|---|---|---|---|---|---|---|---|
| | 60 | 120 | 180 | 240 | 300 | 360 | 390 | 420 | 440 | 460 | 480 |
| % released | 0 | 0 | 0 | 0 | 0 | 0 | 0 | 0 | 3 | 88 | 100 |

Clearly, no leakage occurred during the first 7 hours. Initial release was recorded after elapse of 7 hours and 13 minutes. A major surge of caffeine concentration was detected between 7 hours 13 minutes and 7 hours 23 minutes. Virtually nothing further was detected beyond 7 hours 46 minutes. These results were consistent with the tactical aim of allowing a nominal interval of sleep prior to release of an arousal agent.

### Example 2

### Amphetamine Mini-Tabs Coated with Ethylene-Vinyl Acetate/Cellulose Nitrate Membrane, Zaleplon Calmative

### Preparation of Mixed Amphetamine Tablet Cores

The active wakeup agent and ancillaries for direct compression are proportioned according to the following table:

| | | | | % | |
|---|---|---|---|---|---|
| | | | | total | Batch |
| | | Subunit | Dose | dose | size, |
| Ingredient: | Grade: | mg: | mg/unit: | wt: | g: |
| d-Amphetamine sulfate | 100% pure | 0.25 | 8.0 | 4.0 | 8.0 |
| Methamphetamine HCl | 100% pure | 0.25 | 2.0 | 1.0 | 2.0 |
| Croscarmellose sodium | Ac-Di-Sol ® | 2.9 | 58.0 | 29.0 | 58.0 |
| Microcrystalline | Ceolus KG | 2.2 | 44.0 | 22.0 | 44.0 |
| cellulose | | | | | |
| Polyvinylpyrrolidone | Povidone | 0.8 | 16.0 | 8.0 | 16.0 |
| | K30 | | | | |
| Maltodextrin | M520 XXX | 1.8 | 36.0 | 18.0 | 36.0 |
| Dicalcium phosphate | >99+% pure | 1.7 | 34.0 | 17.0 | 34.0 |
| Na stearyl fumarate | Pruv ®, | 0.1 | 2.0 | 1.0 | 2.0 |
| | <10μ | | | | |

| | | | | |
|---|---|---|---|---|
| Total | 10.0 | 200.0 | 100.0 | 200.0 |

A quantity of 2.0 grams of the lubricant, sodium stearyl fumarate (Pruv®, Penwest) was screened through a #80 mesh sieve. The active agents, 2.0 grams methamphetamine HCl (Mission Pharmacal) and 8.0 g d-amphetamine sulfate (Smith K Beecham) were each ground in a benchtop mill (FitzMill® L1) until homogeneous. The active agents were then combined with 58.0 grams of croscarmellose sodium (Ac-Di-Sol® SD-711; FMC), 44.0 grams of microcrystalline cellulose (Asahi Kasei), 16.0 grams of Povidone K30 (AAA Int.), 36.0 g maltodextrin (Maltrin®; Grain Processing Corp.), and 34.0 g of dicalcium phosphate dihydrate (Clarkson CP) in a V-blender and mixed for 10 minutes. The final ingredient, Pruv®, was added and the entire batch blended for another 8 minutes. Subsequently, the mix was charged into a single-punch tabletting machine (Manesty E2, for R&D) equipped with a 2.75 mm socket-type die and spherical punch, and compressed with 1.75 tons. After compression of the powder, the gross yield was about 20,000 mini-tablets each weighing 10 mg. The product was inspected visually and approximately 170 imperfect cores were removed. Furthermore, an identical batch was run except for substitution of 10.0 grams of amaranth dye instead of the active agent.

### Formation of E.V.A./C.N. Membrane by Application in Wurster Column

The solids are formulated as in the following table:

| | | Subunit | Dosage | | |
|---|---|---|---|---|---|
| | | Wt., | Wt., | Layer | Batch |
| Ingredient: | Grade/Type: | mg: | mg: | % wt.: | Size, g: |
| Ethylene-vinyl | Elvax 40W | 0.33 | 6.6 | 51.6 | 5.94 |
| acetate | | | | | |
| Cellulose nitrate | M, Med. visc. | 0.30 | 6.0 | 46.8 | 5.40 |
| Diethylene glycol | K-Flex ® DE | 0.01 | 0.2 | 1.6 | 0.18 |
| dibenzoate | | | | | |

| | | | | |
|---|---|---|---|---|
| Subtotals, coating solids | 0.64 | 12.8 | 100.0 | 11.52 |
| Compressed Cores | 10.00 | 200.0 | | 180.00 |
| Total weight of coated cores | 10.64 | 212.8 | | 191.52 |

Five and ninety-four hundredths (5.94) grams of ethylene-vinyl acetate copolymer (duPont Elvax 40W), 5.40 grams cellulose nitrate (ICI Nobel), and 0.18 gram (1.6%) of the plasticizer, diethylene glycol dibenzoate (BFGoodrich Kalama), were dissolved in a carefully balanced organic solvent system.

The solvent blend was composed as per the table below:

| Solvent: | Volume, %: | Volume, ml: |
|---|---|---|
| Butyl acetate | 12 | 54 |
| ethyl acetate | 22 | 99 |
| 1-Butanol | 26 | 117 |
| Methyl ethyl ketone (MEK) | 18 | 81 |
| Toluene (diluent) | 22 | 99 |
| Totals | 100 | 450 |

A Glatt CPCG-3, with High Speed (HS) Wurster insert, is loaded with 180 grams of mini-tablet cores prepared by direct compression as above. The nozzle was positioned at the bottom, and the bed was preheated to 45° C. Processing was conducted with parameters according to the following table:

| | Time, minutes | | | | | |
|---|---|---|---|---|---|---|
| | 0 | 10 | 20 | 30 | 40 | 50 |
| Inlet temp., ° C. | 65 | 68 | 70 | 70 | 60 | 45 |
| Product temp., ° C. | 48 | 50 | 50 | 50 | 40 | 36 |
| Outlet temp., ° C. | 42 | 40 | 35 | 35 | 32 | 30 |
| Spray rate, binder, g/min | 9 | 10 | 10 | 10 | 8 | — |
| Atomiz. pressures, bar | 1.8 | 2.0 | 2.0 | 2.0 | 1.8 | — |
| Air flow rate, m³/hr | 170 | 185 | 185 | 185 | 180 | 175 |

The coating operation was continued until all of the solution had been applied and fluidization was then maintained at lower rate while the film dried. The yield was approximately 18,000 coated mini-tablets, essentially all of which were of acceptable quality. The mean membrane thickness was 28 microns. Likewise, the batch of cores having amaranth dye was coated by the same process.

### Application of Prompt-Release Zaleplon Calmative Layer

Proportions for dry matter were as per the following table:

| | | | Dose | Layer | |
|---|---|---|---|---|---|
| | | Subunit | Wt., | Wt., | Batch |
| Ingredient: | Grade/Type: | wt., mg: | mg: | %: | Wt., g: |
| Zaleplon | 100% | 0.25 | 5.0 | 29.0 | 4.25 |
| Methylcellulose | Methocel E-5 | 0.20 | 4.0 | 23.3 | 3.40 |
| Methylcellulose | Methocel E-15 | 0.20 | 4.0 | 23.3 | 3.40 |
| Polyethylene Glycol | 3350 NF | 0.15 | 3.0 | 17.4 | 2.55 |
| Polyvinylpyrrolidone | Plasdone ® | 0.06 | 1.2 | 7.0 | 1.02 |
| | C-15 | | | | |

| | | | | |
|---|---|---|---|---|
| Subtotals | 0.86 | 17.2 | 100.0 | 14.62 |
| Previously prepd cores | 10.64 | 212.8 | | 180.88 |
| Total | 11.50 | 230.0 | | 195.50 |

Because the calmative agent, zaleplon (WyethLederle/AHP) is practically insoluble in water, the chosen solvent blend was an aqueous solution of 80% ethanol. The application was by spray in the HS Wurster column, with procedure nearly identical to that for the calmative layer in Example 1.

The completed batch consisted of about 17,000 coated mini-tablets, with diameter of 2.8 mm each, deviation being negligible. The quantity filled 900 size #00 standard gelatin capsules with 20 subunits each.

The second batch, having amaranth dye in the cores, was overcoated by the same routine, except with substitution of FD & C Blue #2 pigment instead of the zaleplon calmative.

### Testing

Testing for release of calmative was conducted as per Example 1, with results coinciding as expected. Release of wakeup agent was tested as per Example 1, except by spectrophotometrically measuring the absorbance of the amaranth dye at 520 nm. No leakage occurred during the first 7 hours. Initial release was recorded at 7 hours and 6 minutes. After elapse of 7 hours and 12 minutes, 6% was detected. A major surge of dye concentration was detected between 7 hours 12 minutes and 7 hours 18 minutes. The remaining complement was apparently released in full by 7 hours 20 minutes.

### Example 3

### Methylphenidate Cores with Cellulose Acetate Membrane, Triazolam Calmative

### Rotogranulation/Powder-Coating of Methylphenidate on Non-Pareil Seeds

The technique is based on build-out of inert seeds, which in this case are sugar spheres and in this specific example are sized somewhat above the typical range. Formulation for the cores is set forth in the table below:

| | | Subunit | Dosage | | |
|---|---|---|---|---|---|
| | | Wt., | Wt., | Layer | Batch |
| Ingredient: | Grade: | mg: | mg: | % wt.: | size, g: |
| Methylphenidate, | Pure, d-threo- | 0.33 | 9.9 | 8.46 | 9.9 |
| HCl | | | | | |
| Microcrystalline | Avicel PH101 | 2.09 | 62.7 | 53.59 | 62.7 |
| cellulose | | | | | |
| Crosscarmelose | Ac-Di-Sol | 1.16 | 34.8 | 29.74 | 34.8 |
| Na | | | | | |
| Subtotals | Plasdone S-630 | 3.58 | 107.4 | 91.79 | 107.4 |
| Povidone | | 0.32 | 9.6 | 8.21 | 9.6 |
| Subtotals | hard, #10 | 3.90 | 117.0 | 100.00 | 117.0 |
| Sugar seeds | | 2.10 | 63.0 | | 63.0 |
| Totals | | 6.00 | 180.0 | | 180.0 |

The subunit cores are prepared by first comminuting and blending the methylphenidate powder with the excipients—microcrystalline cellulose (Avicel® PH101, FMC Corp.), and sodium carboxymethylcellulose (FMC Corp.) in a spiral jet mill (Atritor Microniser model 2) to a particle size less than 5 microns. The fluid bed apparatus is an upgraded Glatt GPCG-3, with rotor processor module. A powder feeding apparatus is also enjoined. After blending, the rotor processor is charged with 63.0 grams of #10 mesh (2 mm), hard sugar beads (E. Castelli, Milan) and the powder feeder with 117.0 grams of the methylphenidate/excipient blend. Next, a 10% polyvinylpyrrolidone (PVP) aqueous binder solution is prepared by mixing 9.6 grams of PVP into a solvent blend of 950 ml ethanol and 50 ml distilled water. The nozzle is positioned for tangential spray.

Powder layering began with preheating via the air inlet at 60° C. until the bed temperature reached 55° C. After activation of the rotor plate, set at 150 rpm, tangential spraying of the PVP solution is commenced onto the sugar spheres. The seeds are sprayed with the binder until they became tacky enough for adhesion of the next ingredients. Then feeding of the powder comprising the drug is initiated. As the powder begins to layer onto the seeds, the rotor speed is increased to 1,000 rpm, and both binder spray rate and powder feed rate are increased.

The machine parameters are as follows:

| | Time, minutes | | | | | | |
|---|---|---|---|---|---|---|---|
| | 0 | 15 | 30 | 45 | 60 | 75 | 90 |
| Inlet temp, ° C. | 60 | 65 | 70 | 68 | 62 | 55 | 45 |
| Product temp., | 55 | 59 | 64 | 62 | 57 | 49 | 37 |
| ° C. | | | | | | | |
| Outlet temp., ° C. | 42 | 44 | 48 | 47 | 43 | 37 | 31 |
| Spray rate, g/min | 8 | 12 | 15 | 15 | 10 | 8 | — |
| Powder feed rate, | — | 1.7 | 2.5 | 2.5 | 1.3 | — | — |
| g/min | | | | | | | |
| Atomiz. pressure, | 1.2 | 2.0 | 2.8 | 2.8 | 2.0 | 2.0 | — |
| bar | | | | | | | |
| Rotor speed, rpm | 150 | 800 | 1000 | 1000 | 250 | off | off |
| Air flow rate, | 170 | 175 | 180 | 180 | 180 | 175 | 165 |
| m³/hr | | | | | | | |

After 60 minutes, the rotor speed, powder feed rate and the application rate of the binder are all reduced. The product bed temperature is maintained in the range of 55-65° C. during the application of the binder and powder. Upon consumption of all powder blend after 75 minutes, the rotor is switched off, while spraying of the PVP solution is continued for about 13 minutes at a further reduced rate. The rotogranulated cores are then dried with reduced air velocity at a product temperature of 35-40° C. for 10 minutes.

The batch is further dried for 6 hours in a conventional oven set at 40° C. to remove solvent. The layered pillets are next passed through a special #8 screen woven of 0.889 mm (0.0350″) wire and having 2.29 mm holes (Mahaveera Industries, Navi Mumbai, India) to remove oversized beads, then through a #9 gauge sieve having 2.2 mm holes (Mahaveera) to collect a uniform product having a concise core size and weight of almost exactly 2.26 mm and 6.0 mg. A duplicate batch is also run, identical to the first except for substitution of 5.0 grams of amaranth dye and 4.9 grams monocalcium phosphate monohydrate (Ajax) as filler instead of the active agent.

The coating apparatus, including the spray nozzle, tanks, pumps, and lines are cleaned thoroughly before the next step.

### Formation of Cellulose Acetate Membrane by Application in Wurster Column

A coating solution is made of cellulose acetate polymer (Eastman Chemical Products, Inc.), with acetyltri-n-butyl citrate (ATBC; Morflex) and acetyltriethyl citrate (ATEC; Morflex) as plasticizers, each in an amount 4.0% of the weight of dry polymer. The polymer and plasticizers are dissolved in a 500 ml solvent system of 35% methyl ethyl ketone, 50% methylene chloride, 10% ethyl acetate, and 5% isopropanol.

Proportions for the solids are as follows:

| | | Subunit | Dose | Layer | Batch |
|---|---|---|---|---|---|
| Ingredient: | Grade/Type | wt., mg: | Wt, mg: | % wt.: | Wt., g |
| Cellulose acetate | 398-30NF | 0.390 | 11.70 | 92.86 | 9.286 |
| Acetyltributyl | Citroflex A-4 | 0.015 | 0.45 | 3.57 | 0.357 |
| citrate | | | | | |
| Acetyltriethyl | Citroflex A-2 | 0.015 | 0.45 | 3.57 | 0.357 |
| citrate | | | | | |

| | | | | |
|---|---|---|---|---|
| Subtotal | 0.420 | 12.60 | 100.00 | 10.000 |
| Active cores | 6.000 | 180.00 | | 142.700 |
| Total | 6.420 | 192.60 | | 152.700 |

The previously prepared methylphenidate-loaded pillets are then spray coated with the polymer solution in the High Speed (HS) Wurster column of the GPCG-3 to form a semipermeable coating. Associated equipment: peristaltic pump, and 90 psi 120 gallon compressed air supply. Preheating is with inlet at 60° C., until the bed reaches 45° C., (approximately 5 minutes). The nozzle is at the bottom position.

Machine parameters are arranged as per the table following:

| | Time, minutes | | | | |
|---|---|---|---|---|---|
| | 0 | 10 | 20 | 30 | 40 |
| Inlet temp, ° C. | 60 | 62 | 65 | 62 | 60 |
| Product temp., ° C. | 45 | 46 | 48 | 46 | 45 |
| Outlet temp. ° C. | 40 | 41 | 42 | 41 | 40 |
| Spray rate, g/min | 10 | 12 | 15 | 13 | — |
| Atomiz. pressures, bar | 2.0 | 2.5 | 2.8 | 2.5 | — |
| Air flow rate, m³/hr | 165 | 170 | 170 | 170 | 150 |

The coating time is 30 minutes. Drying is allowed at reduced airflow for 10 additional minutes, at a product temperature of 30-35° C. The gross of 20,000 coated subunits is first passed through another special sieve, #8 mesh, woven of 0.812 mm wire (0.0320″), and having 2.36 mm holes (Newark Wire Cloth), to remove agglomerates and oversizes. Then the batch is screened with the same #8 sieve as used above (2.29 mm holes) to remove particulates. Approximately 3,800 subunits, massing 25 g, are rejected as imperfections. The final yield is about 16,200 subunits weighing 6.5 mg each with average core diameter of 2.32 mm. The cellulose acetate membrane is approximately 23 microns thick. Also, the batch having amaranth dye is coated by exactly the same routine.

### Application of Prompt-Release Triazolam Calmative Layer

The formulation for the calmative layer is set forth in the table below:

| | | | Dose | | |
|---|---|---|---|---|---|
| | | Subunit | Wt., | Layer | Batch |
| Ingredient: | Grade/Type | wt., mg: | mg: | Wt. %: | Wt., g |
| Triazolam | 100% pure | 0.004 | 0.12 | 0.51 | 0.06 |
| (Halcyon) | | | | | |
| Methylcellulose | Methocel | 0.360 | 10.80 | 46.16 | 5.40 |
| | E-5 | | | | |
| Methylcellulose | Methocel | 0.366 | 10.98 | 46.92 | 5.49 |
| | E-15 | | | | |
| Polyethylene | PEG 4000 | 0.050 | 1.50 | 6.41 | 0.75 |
| glycol | NF | | | | |

| | | | | |
|---|---|---|---|---|
| Subtotals | 0.780 | 23.40 | 100.00% | 11.70 |
| Previously coated cores | 6.420 | 192.60 | | 96.30 |
| Totals | 7.200 | 216.00 | | 108.00 |

The coating material employed for the calmative layer is a mixture of “Methocel E-5 Premium” and “Methocel E-15 LV Premium” (Dow Chemical Company), USP grades. Active agent, polymers, and plasticizer are dissolved in 1,100 ml of solvent composed of equal parts of distilled water and ethanol. The solids are portioned as 0.06 grams triazolam (Pharmacia & Upjohn), 5.4 grams Methocel E-5, 5.49 grams Methocel E-15, and polyethylene glycol 4000 USP (Farma International) in the amount of 0.75 grams as a plasticizer to provide about 10% total weight by weight solids in solution.

Spray application is conducted in the HS Wurster column with components of the Glatt GPCG 3 apparatus configured as in the previous step, including nozzle position at bottom. The bed is preheated until it reaches approximately 38° C. Parameters during the calmative layer application step are as follows:

| | Time, minutes | | | | |
|---|---|---|---|---|---|
| | 0 | 20 | 40 | 60 | 80 |
| Inlet temp, ° C. | 55 | 57 | 58 | 58 | 50 |
| Product temp., ° C. | 37 | 38 | 39 | 40 | 35 |
| Outlet temp. ° C. | 29 | 30 | 32 | 32 | 28 |
| Spray rate, g/min | 8 | 12 | 15 | 15 | — |
| Atomiz. pressure, bar | 1.2 | 2.0 | 2.8 | 2.8 | — |
| Air flow rate, m³/hr | 165 | 175 | 175 | 175 | 165 |

Spray time is 80 minutes. After coating is completed, the subunits are dried at reduced airflow for 10 minutes with the air temperature at 40° C. and then for five minutes with the air heater off.

After agglomerates and fine powder are separated, the net is 97.2 g, or about 13,500 spherical pillets having an average weight of 7.2 mg and a built-up diameter of 2.4 mm, for 450 #0 standard gelatin capsules, 30 subunits each.

The batch having amaranth dye in the cores is overcoated by the same process, except with substitution of FD & C Blue #2 pigment instead of triazolam. The revised proportions are shown in the following table:

| | | | Dose | | |
|---|---|---|---|---|---|
| | | Subunit | Wt., | Layer | Batch |
| Ingredient: | Grade/Type | wt., mg: | mg: | Wt. %: | Wt., g |
| FD & C dye | Blue # 2 | 0.07 | 2.1 | 8.97 | 1.05 |
| Methylcellulose | Methocel | 0.32 | 9.6 | 41.03 | 4.80 |
| | E-5 | | | | |
| Methylcellulose | Methocel | 0.34 | 10.2 | 43.59 | 5.10 |
| | E-15 | | | | |
| Polyethylene | PEG 4000 | 0.05 | 1.5 | 6.41 | 0.75 |
| glycol | NF | | | | |

| | | | | |
|---|---|---|---|---|
| Subtotals | 0.78 | 23.4 | 100.00% | 11.70 |
| Previously coated cores | 6.42 | 192.6 | | 96.30 |
| Totals | 7.20 | 216.0 | | 108.00 |

### Testing:

Testing for release of calmative is conducted as per Example 1, with mirrored results.

Release of wakeup agent is tested as per Example #1, except by spectrophotometrically measuring the absorbance of the amaranth dye at 520 nm. Again, results mirror those of Example 1.

What is claimed is:

1. A sleep regulating system relating to states of consciousness and comprising a pharmaceutical formulation which effects transitions between said states, said formulation generally featuring an initial release which is compatible with onset of drowsiness and slumber, a subsequent delay phase corresponding to a nominal interval of sleep, and a conclusional arousal release promoting reversion to consciousness with ensuant alertness and vigor during early activities, the formulation having a dosage form designed for oral administration before bedtime.

2. The sleep regulating system according to claim 1 organizationally configured such that the initial transition, from waking to sleep, is effected by an outer component group, and both the delay phase and reversion to the conscious state are accomplished by an inner subsystem.

3. The sleep regulating system according to claim 2 prepared as a dosage form in which delay of release is effected by any of the various means known to the pharmaceutical art, either as single methods or as combinations thereof.

4. The sleep regulating system according to claim 2 wherein the outer component group is normally comprised of at least one layer, said layer or layers carrying one or more sleep-compatible substance which is generally arranged for prompt release, and wherein the inner subsystem includes at least one subunit, said subunit being comprised of a coated core, said core containing at least one active wakeup agent and completely enveloped by a coating distinct from any outer component, said coating fabricated from at least one film-forming material and constituting an osmotic semipermeable membrane.

5. The sleep regulating system according to claim 4 wherein the osmotic semipermeable membrane is characterized by ample but finite elasticity, pharmaceutical inertness, insolubility in aqueous media, permeability to water, impermeability to outward flux of active agent, and the behavior of said membrane, pursuant to ingestion of the dosage form, is progressive expansion by internal pressure in synchronous correlation with residence in the gastrointestinal tract until the membrane bursts, thereby accomplishing delayed release of the wakeup agent.

6. The sleep regulating system according to claim 5 comprised such that delay is effected by a combination of means or subsystems, including, but not restricted to, semi-permeable membrane rupture after a definite time period, wherein a main membrane surrounds an agglomeration of smaller subunits such as pellets and granules, and optionally said smaller subunits are individually coated by membranes.

7. The sleep regulating system according to claim 4 wherein the semipermeable membrane features at least one weak spot which, pursuant to absorption of water by the core, ruptures primarily at said weak spot resulting in release of the agent to the gastrointestinal tract, the length of delay from administration to said rupture being at least partially determined by the degree of reduction of strength of the weak spot relative to strength of the other areas of the membrane.

8. The sleep regulating system according to claim 4 wherein the semipermeable membrane features at least one seam which, pursuant to absorption of water by the core, ruptures primarily along said seam resulting in release of the agent to the gastrointestinal tract, the length of delay from administration to said rupture being at least partially determined by the relatively deficient bond of the seam as compared to the strength of other areas of the membrane.

9. The sleep regulating system according to claim 4 wherein the principal materials of the semi-permeable membrane may be selected from the group of polymers comprised of organic cellulose esters such as cellulose acetate; inorganic cellulose esters such as cellulose nitrate; cellulose ethers such as ethylcellulose; polyvinyl alcohols; polyurethanes; vinyl esters such as polyvinylacetate and ethylene vinyl acetate; and polyacrylics; but said materials not restricted to selection only from said group, and eligible for any compatible combination as well as single-material compositions.

10. The sleep regulating system according to claim 4 designed with a plurality of subunits, cores of the subunits formed as pellets, beads, or mini-tablets, each subunit having its own membrane and outer sleep-compatible layer, all of the individual subunits prepared so as to release their contents simultaneously and aggregated by conventional methods such as, but not limited to, filling into capsules and pressing into shaped bodies, and wherein optional arrangements for the outer sleep-compatible component may include powder loading into said conventionally filled capsules, coating of the outer surfaces of conventional capsules, and superposition onto said shaped bodies by the various means known to the art.

11. The sleep regulating system according to claim 4 wherein the sleep-compatible substance is any selection from the group comprised of tonics, calmatives, hypnotics, muscle relaxants, sedatives, anti-anxiety agents, anti-insomnia agents, tranquilizers, neutral materials having no pharmaceutically active character, hormones, endorphins, herbal preparations, and substances traditionally reputed to have soporific effects, such as but not confined to: benzodiazepines including lorazepam, temazepam, triazolam, their derivatives and close relatives; non-benzodiazepines represented by zaleplon, zolpidem tartrate, L-tryptophan, 5-hydroxy-L-tryptophan, melatonin, sleep-promoting factor, their derivatives, and close relatives; drugs whose availability has been postponed due to detentions such as but not limited to further development, ongoing clinical trials, and FDA review, including eszopiclone which is also known as S-Zopiclone, further including but not restricted to NGD 91-2, NGD 96-3, and NS2710, in addition to indiplon which is also known as NBI-34060.

12. The sleep regulating system according to claim 4 wherein at least one wakeup agent is selected from the group comprised of pharmaceutically active energizers, invigorants, nervous system stimulants, and psychostimulants, including but not restricted to amphetamines, methylphenidate, venlafaxine, nefazodone, sodium oxybate, adrafinil, modafinil, phentermine, pemoline, adrenaline; methyl xanthines including theophylline, theobromine, and caffeine; substances pending release due to further development, clinical trials, FDA review, or other detention; drugs temporarily withdrawn from availability; as well as close relatives and derivatives of members from said group; all members being eligible for any compatible combination.

13. The sleep regulating system according to claim 4 wherein pharmaceutical agents for treatment of Attention Deficit Disorder and Attention Deficit Hyperactive Disorder comprise a group from which at least one substance is adapted as a wakeup agent.

14. The sleep regulating system according to claim 4 wherein the core may include any appropriate conventional excipients known to the art, including gas-generating substances such as, but not restricted to, sodium bicarbonate and calcium carbonate coupled with one or more mild acids such as citric acid and sodium dihydrogen phosphate.

15. The sleep regulating system according to claim 4 wherein the length of the delay between administration of the dosage form and release of the arousal agent can be programmed by any combination of technical methods comprised from: discretionary selection of specific materials for fabrication of the semipermeable membrane, change of the thickness and thus the permeability of the membrane, supplementation of the core with osmotic attractant, change of the surface area of the subunit, and alteration of the overall dimensions of the subunit, said alteration effected by means such as modification of the radius.

16. A method of preparation for the sleep regulating system according to claim 4 wherein the subunit cores are fashioned by established, improved, or new pharmaceutical techniques including, but not restricted to, formation as rounded granules by rotor granulation, small tablets by direct compression, and spheres, possibilities for origination of said spheres including colloid gelatination, rounding of extrudates, direct pelletization, and buildup unto pre-fabricated inert bodies such as sugar balls.

17. A method of preparation for the sleep regulating system according to claim 4 in which the inner subsystem is comprised of a prefabricated time-disintegration tablet or caplet, said tablet or caplet bored to form a hollow space, filled with an active wakeup compound, and re-sealed with a material which effectively restores the integrity of the dosage form and re-enables its delayed release function.

18. The sleep regulating system according to claim 4 wherein at least one portion of substance is scheduled for release during the delay phase before release of the wakeup agent, said substance selected from the group including but not restricted to complementary therapeutic agents, nutrients, and sleep-assisting agents, options for the character of the substance being identical to, similar to, and different from any initially released agent.

19. The sleep regulating system according to claim 18, whereby means for interim release of substance may include, but are not restricted to, multiple semipermeable membranes configured to burst in series, the innermost of said membranes being responsible for containment and ultimate release of the wakeup agent.

20. The sleep regulating system according to claim 1 wherein the lag time between administration and the start of arousal action is, in basic embodiments, optimally in the range of about 5 to 9 hours and preferably about 5 to 7.5 hours.

21. The sleep regulating system according to claim 1 arranged for a short delay, wherein the nominal interval of sleep is a nap, and thus the lag between administration and the start of stimulus action is optimally in the range of about 2 to 5.5 hours, particular applications for such short delay formulations including, but not restricted to, therapy for incontinence.

22. The sleep regulating system according to claim 1, wherein the initial release is substituted by a phase featuring no release of a pharmaceutically active agent.

23. The sleep regulating system according to claim 4 wherein, pursuant to absorption, persistence of the wakeup agent as residual in the bloodstream during elimination enables alertness and vigor in early activities.

24. A method for reducing tardiness and absenteeism, comprised of the pharmaceutical formulation according to claim 23, whereby positive transition from sleep to wakefulness, assisted by protracted early vigor, constitutes a strong start, said strong start promoting punctual arrival at destinations such as school and employment, and sharply reducing the chance that susceptible individuals might not attend said destinations.

25. The method of claim 24, suited for students, options for administration including by self and by parent, and further options including provision by schools.

26. The method of claim 24, purposed to reinforce employment stability of a worker, and applied via self-administration.

27. The method of claim 24, arranged for an employer, wherein the pharmaceutical formulation is made available by said employer to employees, benefits to the employer upon usage by said employees including, but not limited to, improved business productivity and relief of economic loss, said benefits derived chiefly from reduced dismissals and thus decreased turnover in personnel.

28. The method of claim 24, projected to moderation of macroeconomic unemployment, benefits upon usage including, but not limited to, decrease in dismissals of workers.

29. A method for treating stress, comprised of administration of the pharmaceutical formulation according to claim 23; indications for said method often including stress manifest by anxiety disorders, and the method further indicated wherein stress is concurrent with depression; benefits of the formulation which may include, but are not restricted to, consistency of initial organization pursuant to wakeup and reinforced employment surety; the method alternately comprised of the sleep regulating system administered in conjunction with other stress therapies, possibilities for said therapies including anxiolytic drugs as well as non-pharmaceutical stress management strategies.

30. A method for treating depression comprised of the pharmaceutical formulation according to claim 23 administered to a patient in conjunction with other anti-depressant therapies, the preparation serving to assist punctuality and productive vigor in early waking activities, benefits of said assistance including, but not limited to, improvement of sense of efficacy, said method optionally comprised of the formulation administered independent of other therapies.

31. A method for treating Chronic Fatigue Syndrome comprised of administration of the pharmaceutical formulation according to claim 23, serving to alleviate symptoms in early activities, said alleviation including compensation for oppressive weariness and inertia, further benefit to afflicted individuals including, but not limited to, assisted punctuality, improved personal safety via reduced probability of drowsing or falling asleep while in transit to work, and correspondingly fortified employment surety, said method optionally comprised of the formulation administered as adjuvant to other therapies which may be available.

32. A treatment for sleep disorders comprised of the sleep regulating system of claim 23, wherein symptoms of said disorders include detrimental patterns such as sequences or circles of sleep inadequacy accompanied by difficulty in awakening at a reasonably scheduled time point, and whereby the system weakens, and, optimally, disrupts, said patterns.

33. A treatment for Insomnia and sleep disorders which include insomnia as a symptom, comprised of the sleep regulating system of claim 23, by which, in addition to initial insomnia being addressed per release of calmative or other sleep-compatible substance, the integral formulation harmonizes the overall sleep cycle, said harmonization accomplished collaboratively by the conclusional release, residual arousal agent from which, pursuant to assisting wakeup, promotes vigor and productivity in early waking activities, the treatment thus reducing compulsions to ingest stimulants later in the day and immerse in agitating engagements before bedtime, and thereby diminishing sources of the insomnia.

34. The treatment according to claim 33 wherein at least one portion of calmative or other sleep-compatible substance is arranged for interim release during the delay phase, said portion addressing middle insomnia, and said interim release arrangement optionally featuring no initial release of sleep-compatible agent.

35. A treatment for sleep disorders principally including, but not confined to, Hypersomnia and Sleep Paralysis, comprised of the sleep regulating system of claim 23, wherein not only is difficulty in making transition from sleep to wakefulness addressed by the conclusional release of arousal agent, but the overall sleep cycle is attuned via the integral formulation, said tuning achieved in some measure per the initial release, the calmative or other sleep-compatible substance from which helps the suffering individual to overcome any incidental initial insomnia as may occur, said insomnia being a possible primary or contributing cause of said principal sleep disorders.

36. The treatment according to claim 35 wherein at least one portion of calmative or other sleep-compatible substance is arranged for release during the delay phase, said portion addressing middle insomnia, the formulation optionally featuring no initial release of sleep-compatible agent.

37. An aid to a motor vehicle operator for driving which is executed early in said operator's day or waking hours, said aid especially valuable to a driver younger than about 31 years old, and comprised of the pharmaceutical formulation according to claim 23 administered prior to sleep which precedes said driving, whereby alertness during vehicle operation reduces probability of involvement of the operator in a traffic accident.

38. A method for reducing traffic accident statistics, based upon the aid of claim 37, wherein the aid is made easily accessible and widely available to motor vehicle operators, through such as, but not restricted to, driver licensing offices, vehicle dealerships, places of employment, and educational institutions.
